(19) United States
(12) Patent Application Publication
(10) Pub. No.: US 2017/0283460 A1
(43) Pub. Date: Oct. 5, 2017

(54) Immune System Modulation for Prophylaxis and Treatment of Diseases and Disorders

(71) Applicant: Marpe Holdings, LLC, Boca Raton, FL (US)

(72) Inventors: Kenneth O. Willeford, Starkville, MS (US); Douglas A. McClain, Boerne, TX (US)

(73) Assignee: Marpe Holdings, LLC, Boca Raton, FL (US)

(21) Appl. No.: 15/478,860

(22) Filed: Apr. 4, 2017

Related U.S. Application Data
(60) Provisional application No. 62/455,166, filed on Feb. 6, 2017.
(60) Provisional application No. 62/453,586, filed on Feb. 2, 2017.
(60) Provisional application No. 62/318,016, filed on Apr. 4, 2016.

(51) Int. Cl. C07K 7/08

(52) U.S. Cl. CPC C07K 7/08; A61K 38/00

(57) ABSTRACT

The invention is directed to biological response modifiers (BRM) which may contain one or more compounds, and to methods for enhancement of an immune system with BRMs of the invention including augmenting a specific immune response either prophylactically or for treatment, as an adjuvant or vaccine when coupled with a pathogenic antigen, and for boosting an immune system generally. The invention is also directed to the reduction of an unrestrained or improper inflammatory response and/or an immune response such as to treat or prevent autoimmune diseases and disorders, and associated symptoms. Further, the invention is directed to the manufacture of BRM compounds comprising isolated serum from a mammal, and subjecting that serum to tangential flow chromatography and molecular weight cut-off dialysis to obtain one or more purified BRM compounds suitable for administration to a patient in need.

### REFERENCE TO RELATED APPLICATIONS

This application claims priority to U.S. Provisional Application No. 62/455,166 filed Feb. 6, 2017, U.S. Provisional Application No. 62/453,586 filed Feb. 2, 2017, and U.S. Provisional Application No. 62/318,016 filed Apr. 4, 2016. The entirety of each application is specifically incorporated by reference.

### SEQUENCE LISTING

The instant application contains a Sequence Listing which has been submitted in ASCII format via EFS-Web and is hereby incorporated by reference in its entirety. Said ASCII copy, created on Apr. 4, 2017, is named 3090.002.US_SL.txt and is 4,077 bytes in size.

### BACKGROUND

### 1. Field of the Invention

The present invention provides compositions and methods for enhancement of an immune system including augmenting a specific immune response either prophylactically or for treatment, as an adjuvant or vaccine, and for boosting an immune system generally and, in particular, for reduction of an unrestrained or improper inflammatory response and/or an immune response such as to treat or prevent autoimmune diseases and disorders. The invention is also directed to methods for the manufacture of biological response modifiers and, in particular, manufacturing via tangential flow chromatography and molecular weight cut-off dialysis.

2. Description of the Background

Discovering agents that potentiate the immune response is a driving force in modern drug research. Bioactive peptides, such as cytokines, chemokines, and cationic peptides, are classes of “relatively” low molecular weight compounds that have shown promise in this area of research. At least nine immuno-defense peptide products are commercially available with annual sales of over $4 billion (Latham, 1999).

The concept of immunostimulation originated in 1907, when spontaneous tumor regression was identified in some patients after an episode of septicemia (Rush and Flaminio, 2000). In human and veterinary medicine, immunostimulant preparations are used primarily for treatment of chronic viral or bacterial infections. In some instances, immunostimulants have demonstrated efficacy as primary or adjunct treatment of neoplastic conditions (Rush, 2001). The proposed mechanism of action of nonspecific immunostimulant preparations is macrophage activation and subsequent release of cytokines that enhance the immune response (Rush and Flaminio, 2000). Prophylactic administration of immunostimulant preparations prior to pathogen exposure can decrease morbidity and mortality associated with acute infection (Rush, 2001).

The idea of treating and/or preventing diseases and disorders with immunstimulation and, more generally, immunoregulation, has developed considerable. In human medicine, immunoregulatory preparations have progressed from crude microbial, viral, plant, and thymic extracts to synthetic viral complexes and chemically defined drugs (e.g., recombinant cytokines). The crude extract preparations induce nonspecific immunoregulatory activity via generalized macrophage activation. The new generation of immunoregulators, such as recombinant cytokines, has selective effects on particular components of the immune system.

Because cells of the immune system circulate through the blood and lymphatic system, serum is a logical place to look for immunoregulators. Only about half of the over 100 serum proteins have been isolated and characterized (de Gruyter, 1997), leaving a variety of proteins and peptides as potential immunoregulators. For example, Caprine Serum Fraction Immunomodulator (CSFI) is a non-adjuvant immunostimulant derived from goat serum (Ansley, Daniel R. Composition and Method for Immunostimulation in Mammals, U.S. Pat. No. 5,219,578; Jun. 15, 1993). CSFI is ill-defined by Ansley but is said to be composed of a mixture of serum proteins and peptides, 67% of which is immunoglobulin. In that patent Ansley describes a method for collecting the immunoglobulin containing fraction of goat serum from non-immunologically challenged goats. That process consisted of precipitation with sodium sulfate followed by dialysis of the re-suspended precipitate in a 30,000 Dalton MW cut-off dialysis membrane to remove salts and other low molecular weight substances. The dialyzed fraction was then shown to be efficacious in the treatment of a wide variety of animal diseases including equine lower respiratory disease, ovine foot-rot, bovine shipping fever, bovine respiratory disease, canine lymphoma, bovine lymphoma, and canine parvovirus.

Hamm established that this immunoglobulin containing fraction of goat serum could be used as an adjunct to conventional antibiotics in the treatment of equine lower respiratory disease. In that study more than twice as many horses (86%) were able to recover in a three-week period when the treatment was augmented with the caprine serum fraction as compared to the control group (35%) that only received antibiotic treatment (Hamm, 2002). This fraction is now marketed in the U.S. under the trade name PulmoClear™ for the treatment of equine lower respiratory disease.

Some immunoregulators derived from one species appear to provide a short-term immunity from pathogenic infections when administered to a different host species. In a recent study, Willeford was able to establish that a fraction of caprine serum, substantially free of immunoglobulins, could confer significant protection to chickens challenged with a terminal dose of Pasteurella multocida when the caprine serum fraction was administered 24 hours prior to the bacterial challenge (Willeford, 2000). Similar results were noted by Parker in mice challenged with Salmonella typhimurium (Parker, 2002).

Immunoregulators have been derived from sources other than animal serum as well. For instance, a variety of immuno-stimulants have been derived from mycobacterial products (Ford, 1986; Werner and Zerial, 1984; Diasio and LoBuglio, 1995). Regressin-V, an emulsion of mycobacterial cell wall fragments, is licensed for the treatment of a variety of neoplasia in animals. A killed suspension of Propiobacterium acnes, Immunoregulin, is licensed for veterinary use in advanced neoplasia as an adjunct to other therapies. Although these products are capable of stimulating the immune system in animals in a non-specific manner and are therapeutically efficacious, they have also been observed to initiate untoward effects such as fever and allergic reactions that arise from the broad spectrum of the immune stimulating action (Kruth, 1998).

Immunoregulators can be divided into three main groups: (a) immuno-suppressive agents; (b) immuno-stimulating agents (e.g., bacillus Calmette-Guerin vaccine); and (c) the remaining immunoregulators, which include biological response modifiers (e.g., colony stimulating factors, interleukins, interferons, and tumor necrosis factors) (Takx-Kohlen, 1992; Molloy et al., 1993). Cytokines are soluble, low molecular weight polypeptides and glycopepetides produced by a broad range of cell types that have suppressive or enhancing effects on cellular proliferation, differentiation, activation, and motility. For the most part, cytokines are not constitutively secreted, but are produced in response to stimulation by infectious agents or their derived products (e.g., endotoxin), inflammatory mediators, mechanical injuries, and cytokines themselves (Kogut, 2000).

Interleukin-1 (IL-1), tumor necrosis factor-alpha (TNF-alpha), and interferon (IFN) are three cytokines that participate in the immune response. IL-1 is involved in the host's response to antigenic challenge and tissue injury, and has been shown to increase the resistance of mice to pathogenic organisms such as Listeria, Escherichia coli, and Candida albicans (Czuprynski and Brown, 1987; Cross et al., 1989; Pecyk et al., 1989). TNF-alpha and delta-IFN increased the resistance of mice to Salmonella typhimurium (Morrissey and Charrier, 1994). Human delta-IFNs have potent antiviral and antiproliferative activities, and are utilized as anticancer and antiviral therapeutic agents (Chang et al., 1999).

Three families of low molecular weight peptides that have immune regulatory properties are the tachykinins, the thymic hormones, and cationic peptides. The tachykinins are a family of closely related short neuropeptides that were initially identified by their activities as neurotransmitters. Tachykinins are now known to mediate such diverse activities as the proliferation of T-cells, release of TFN-gamma, TNF-alpha, IL-1 and IL-6, and enhanced secretion of immunoglobulins (Maggio, 1990; Eglezos et al., 1991).

The thymic hormones are a family of proteins and peptides whose exact biological role is unknown. They are known to participate in the regulation and differentiation of thymus-derived lymphocytes and have been shown to act like cytokines. Some thymic hormones have been shown to reconstitute defective cell-mediated immunity in patients with various neoplastic diseases and secondary immune deficiencies as a result of chemo- and/or radiotherapy (thymic humoral factor) as well as enhance the production of IL-1, IL-2, TNF-gamma and TNF-alpha (thymosin fraction 5) (Cohen et al., 1979; Dardenne and Savino, 1990).

Some cationic peptides have been observed to initiate an immunostimulant response. A decameric peptide was shown to impede the growth and spreading of established tumors (Folkman, 1999). Other peptides promote antibacterial, antifungal, antiviral, and even wound healing properties (Sanglier et al., 1993; Mizuno et al., 1995; Hancock, 1999). It is believed that these “defense” peptides are more general in their actions than antibodies, and as such, have a broader range of activity (Hancock, 1999). U.S. Pat. No. 7,358,044 identified a factor referred to as immune cell proliferating factor or ICPF. ICPF was defined as 1-peptidyl-(2,3)-diacyl-O-glycerol lipopeptide. The general structure was stated to comprise a nonapeptide with an arginine at the amino terminus, a phenylalanine at the carboxyl terminus, a serine-o-fatty acid ester at the second amino acid of the nonapeptide, and up to a total of three long chain fatty acids, one of which is an unsaturated fatty acid. The fatty acids contained in the ICPF molecule included stearic acid, arachidic acid, and arachadonic acid. This structure was later determined to have no immune stimulating activity (K.O. Willeford, unpublished study).

Accordingly, there is a need for more and well defined immunoregulators for the treatment and/or prevention of diseases and disorders.

### SUMMARY OF THE INVENTION

The present invention overcomes the problems and disadvantages associated with current strategies and designs, and provide new tools, new compositions and new methods for immunoregulation.

One embodiment of the invention is directed to compositions that comprise one or more compounds that provide for immunoregulation of a mammalian immune system. Preferably the one or more compounds are isolated from caprine serum or synthesized genetically (recombinantly) or de novo. Also preferably, the one or more compounds are chemically and/or structurally modified. Preferably the compositions are pharmaceutical compositions containing one or more pharmaceutically acceptable agents. Diseases and disorders include bacterial, viral, fungal and/or parasitic infection, a neoplastic growth such as cancer, or autoimmune diseases.

Preferably the composition comprises pharmaceutical composition containing the peptide sequence of SEQ ID NO 1 and/or SEQ ID NO 4, and no other fibrin peptide sequences. Preferably one or more of the peptide sequences are modified, and preferably the modification is a sulfation and/or a phosphorylation of the first and/or second tyrosine of SEQ ID NO 1 and/or SEQ ID NO 4. Preferably the sequence of the composition is synthetic and unmodified by either lipids or saccharides. Preferably the composition is in a therapeutically effective amount, which is preferably from about 0.1-100 μg/ml. Preferably the pharmaceutically acceptable carrier and preferably the pharmaceutically acceptable carrier is water, oil, edible oil, fatty acids, lipids, polysaccharides, cellulose, glycerin, glycol, and combinations thereof, and is an aqueous and formulated for intravenous administration. Preferably the composition is nontoxic and generates no side effects after administration to a patient.

Another embodiment of the invention is directed to compositions comprising the one or more compounds of the invention in combination with existing compositions and strategies for the treatment and/or prevention of diseases and disorders. Preferably the methods involve isolation of the one or more compounds from caprine or another mammalian serum, and preferably further purification is preferably via tangential flow chromatography and molecular weight cut-off dialysis. A preferred method of manufacturing the pharmaceutical composition of the invention comprises: providing a mammalian serum that is sterile; mixing equal parts of the sterile serum with a sterile 0.5% saline solution to form a mixture; passing the mixture through tangential flow chromatography followed by molecular weight cut-off dialysis and filtration through a 0.2 μm filter forming a filtered solution; apportioning the filtered solution into single dose vessels; and freezing the filtered solution at minus 10° C. or less. Preferably the molecular weight cut-off dialysis is 10 kDa cut-off dialysis. Also preferably the single dose vessels comprise from about 0.5-5 ml each, and the single dose vessels comprises from about 0.1 μg/ml to about 100 μg/ml of a peptide of the sequence of SEQ ID NO 1 and/or SEQ ID NO 4. Preferably the filtered solution is tested for the presence of endotoxin, and the method is performed under GMP standards for pharmaceutical compositions.

Another embodiment of the invention is directed to methods for the treatment and/or prevention of diseases and disorders. Compositions of the invention contain a therapeutically effective amount of one or more compounds and are administered to patients in need thereof. Patients may be any mammal such as a human. Administration may be by parenteral or nonparenteral means, but is preferably intravenous, subcutaneous, intraperitoneal, or oral. Treatment may be for short periods of time (e.g., pulsed) or continuous for long periods or throughout the lifetime of the patient. Preferably, the method of the invention comprising treating a disease or disorder of a patient comprised of the repeated administration of an aqueous pharmaceutical composition comprising the peptide sequence of SEQ ID NO 1 and/or SEQ ID NO 4, and no other fibrin peptide sequences. Preferably the disease or disorder comprises a viral, bacterial, fungal or parasitic infection, or an inflammatory disorder, and the patient is a human or other mammal. Preferably the therapeutically effective dose comprises about 0.5-5 ml containing from about 0.1 μg/ml to about 100 μg/ml of the peptide sequence. Preferably the peptide sequence is modified, and the modification is a sulfation and/or a phosphorylation of the first and/or second tyrosine of SEQ ID NO 1 and/or SEQ ID NO 4. Preferably the sequence is synthetic or otherwise not natural, and the sequence is unmodified by either lipids or saccharides. Preferably repeated administration comprises weekly administration for 2-20 weeks or longer. Preferably administration initiates an immune system cascade and preferably that cascade comprises: upregulation of TNF-α within about 0.5-12 hours after administration; upregulation of IL-6 subsequent to upregulation of TNF-α; and upregulation of IL-10 subsequent to upregulation of IL-6, which down regulates IL-6. Preferably IL-6 is upregulated 6 hours after administration, and IL-10 is upregulated 24 hours after administration. Preferably the method further comprises the upregulation of macrophages, NK cells, INF-gamma, MCP-1, T-cells, B-cell, and/or GM-CFS after the upregulation of IL-6. Preferably the disease or disorder is a malfunctioning immune system which normalizes for the patient after the repeated administrations, and the malfunctioning immune system is an uncontrolled inflammatory response. Preferably CD4 and CD8 levels are elevated in the patient and administration of the aqueous pharmaceutical composition returns CD4 and CD8 levels to a homeostatic level for the patient.

Another embodiment of the invention is directed to compositions of the invention as vaccines and/or vaccine adjuvants for the treatment and prevention of diseases and disorders. Compositions of the invention containing BRMs are useful directly as vaccine adjuvants, and, when coupled with a specific antigen, preferably an antigenic portion of a pathogen that generates an immune response, as vaccines themselves.

Another embodiment of the invention is directed to methods for regulating an immune system by the administration of one or more compounds of the invention. Preferably regulation increases the immune response to an infectious agent, such as a bacterial or viral infection, or decreases an improperly elevated immune response, such as in auto-immune disorders.

Other embodiments and advantages of the invention are set forth in part in the description, which follows, and in part, may be obvious from this description, or may be learned from the practice of the invention.

### DESCRIPTION OF THE FIGURES

FIG. 1A Sample 2 showing full MS spectrum of the two primary components at m/z 1103 and 1131, with the peak at 1109 likely a neutral loss of a water molecule.

FIG. 1B UPLC-MS trace of Sample 2 wherein compound 1103 elutes at 7.48 min. and compound 1131 elutes at 7.74 min. Relative intensities are calculated from the areas under the curves of the peaks.

FIG. 1C Fragmentation spectrum (MS/MS) of compound 1103 of Sample 2.

FIG. 1D MS/MS spectrum of compound 1130 of Sample 2 showing partial mass overlap between two plots up to m/z 997 which indicates overlap from either the N-terminal or the C-terminal ends.

FIG. 1E Compounds 1103 and 1131 as observed in Sample 3.

FIG. 1F Compounds 1103 and 1131 as observed in Sample 4.

FIG. 1G Compounds 1103 and 1131 as observed in Sample 5.

FIG. 1H Compounds 1103 and 1131 as observed in Sample 6.

FIG. 1I Compounds 1103 and 1131 as observed in Sample 1, although fragmentation spectrum indicates otherwise.

FIG. 1J Compounds 1103 and 1131 as observed in other samples.

FIG. 1K Fragmentation spectra of compound 1130 from Sample 7 showing overlap but differences from Sample 1 likely due to a matching amino acid composition but a different sequence order at one or more positions.

FIG. 1L Compound m/z 1103 of Sample 7.

FIG. 1M Fragmentation spectra of compound 1103 of Sample 7.

FIG. 2A Blood profile of patient neutrophils abs.

FIG. 2B Blood profile of patient white blood cell counts.

FIG. 2C Blood profile of patient CD4 cell counts.

FIG. 2D Blood profile of patient CD8 cell counts.

FIG. 2E Blood profile of patient immunoglobulin levels.

FIG. 2F Blood profile of patient immunoglobulin levels.

FIG. 2G Blood profile of patient immunoglobulin levels.

FIG. 2H Blood profile of patient IGG subfraction levels.

FIG. 2I Blood profile of patient IGG subfraction levels.

FIG. 2J Blood profile of patient procalcitonin levels.

FIG. 2K Blood profile of patient C-reactive protein levels.

FIG. 3A Comparison of CD4 and CD8 levels of a first patient.

FIG. 3B Comparison of CD4 and CD8 levels of a second patient.

FIG. 4 Cytokine levels at various times after administration of NPIS40.

FIG. 5A Summary of patient blood chemistry (part A).

FIG. 5B Summary of patient blood chemistry (part B).

### DESCRIPTION OF THE INVENTION

Immuno-therapeutic agents provide for stimulation of an immune system in response to a disease or disorder. Although cytokines such as interferons and TNF often provide dramatic therapeutic results, cytokines also require therapeutic dosages at concentrations that produce toxic side effects that are sometimes fatal and/or not well tolerated. As such these agents are not good candidates for general application. Moreover, it is often an interaction of multiple cytokines that are required to illicit a beneficial effect, which often does not occur with the administration of a single cytokine.

The mammalian immune system is a coordinated and highly organized system of interactions that provide an organism with the ability to adaptively react to nearly all conditions. The essential components of those interactions are cytokines which are responsible for directing a diverse array of functional events within a cell. Some success at arresting cytokine targets have proven to be of therapeutic benefit, but treatments wherein cytokines are directly administered to a patient have shown only moderate success and, therefore, are typically considered only after conventional therapies prove ineffective. Nevertheless, to be able to harness such signal mediators would be an attractive strategy for both therapeutic and prophylactic purposes.

Biological Response Modifiers (BRMs) have been surprisingly discovered that, when administered to a patient, initiates a systemic change in the cytokine profile. The systemic change may be an increase, a decrease or a stabilizing of the relative level of one or more cytokines of the patient's immune system. The terms increase, enhance or activate, decrease, de-enhance or deactivate, stabilize and the like are all used herein to generally mean an increase or decrease, as appropriate, or stabilization by a statistically significant amount as compared to a reference or control level.

It was surprisingly discovered that a cytokine cascade initiated by exposure to a BRM of the invention modulates the innate and/or adaptive immune systems and thereby therapeutically or prophylactically reduced morbidity and mortality in animal models. In chronic inflammation (e.g., hyper and hypo chronic conditions) and also certain diseased states, the immune system is hindered or shut down. Upon exposure to a BRM of the invention, normal functioning of the immune systems is restored through a selective cytokine cascade not otherwise found naturally. The cascade stimulated by the invention is initiated through cellular and hormonal messaging systems that allow the modulating and regulatory actions of cytokines, growth factors, T cells, B cells, MHC-1 and II, in an endogenous manner verses and exogenous manner, thereby affectively re-establishing the cellular actions toward normal or what constitutes normal for the particular patient. A specific part of the immune cascade of the invention, where IL-6 up-regulates, is followed by up-regulation of IL-10, which is hindered or shut down in challenged patients which may be due to uncontrolled inflammatory cytokines that inhibit or stop normal immune protections and can evolve to auto immune responses, loss of the immune protections of anti-viral and anti-bacterial functions of the body as well as non-recognition of mutations or improper cytokine expression which can lead to various forms of cancer (e.g., tumorigenesis, metastasis, etc.). This is then followed by the body's inability to destroy those cells and a state of diagnosable cancer appears. With the endogenous up regulation of IL-10 by the BRM of the invention, the action of the uncontrolled pro inflammatory cytokines, that are the genesis of disease, are reversed.

A preferred cascade initiated by administration of the compounds of the invention comprises the up-regulation of TNF-α, followed within 24-48 hours with the up-regulation of IL-6 (a pro-inflammatory cytokine), followed within 48-96 hours by the up-regulation of IL-10 (an anti-inflammatory cytokine), which in addition to its positive effects, suppresses IL-6. This sequence of events controls and modulates the immune system and prevents the possibility of an uncontrolled cytokine storm. Subsequent to IL-10 expression, there is an un-regulation of TNF-gamma that promotes an anti-viral and/or anti-bacterial cellular immune response, angiogenesis, and also cellular healing. The up regulation of IL-6 (pro-inflammatory cytokine) is controlled by a delayed release of IL-10 (an anti-inflammatory cytokine). Such timed release allows the protective cascades of the innate immune system to be enacted (enhanced protection against bacterial assault) while also eliminating the possibility of a cytokine storm as seen via administration of exogenous cytokines. The delayed release of IL-10 also minimizes chronic inflammation.

One advantage of the compositions and methods of the invention is a return to true cytokine and/or cellular modulation and not simply single agent or cellular suppression or stimulation. A normally successful response to a challenge to the innate immune system is to up-regulate TNFα followed by up-regulation of IL-6. In such a response, when an agent that stimulates IL-6 is administered, the IL-6 response is not regulated, simply increased. The result is an overexpression that produces an inflammatory condition. In those situations, where IL-6 is properly up regulated, administration of the BRM of the invention will not up-regulate or interfere with that process. However, in a challenged patient whose innate immune system is not responding, administration of the BRM of the invention will initiate the proper response of TNFα followed by IL-6 followed by IL-10 48 to 96 hours later. This reestablishes proper modulation of the innate system. Endogenous modulation through BRM administration ultimately helps restore normal homeostasis while eliminating the negative side effects associated with cytotoxic pharmaceuticals. Pharmaceuticals designed to suppress immune cascades (even in a targeted fashion) leave the body at risk.

Chronic inflammation hinders or stops proper cell signaling. An exogenous administration of IL-10 often stops that process, but the inability of administrating a proper dose of IL-10 and patient to patient variability have kept this from being an acceptable choice. Differences exist between individual patients that cannot be assessed within the time period for the course of a treatment. As the invention up-regulates IL-10 in an endogenous method, there is no negative side effect, and the chronic inflammation is reversed with a return to proper cell signaling. The cascade initiated by the invention restores the immune system to homeostasis preventing the possibility of negative side effects produced by single action pharmaceuticals, which block or suppress an immune cascade by interfering with one cytokine leaving the body defenseless against a challenge, or improperly stimulating an immune cascade leading to autoimmune reactions.

Upon administration of a BRM of the invention, within 30 minutes to an hour, TNFα up regulates initiating the release of IL-6 beginning the endogenous Immune cascade. This is preferably followed by up regulation of macrophages and then NK cells. At 12 to 30 hours post administration, a multifold up regulation of IL-10 brings down the pro inflammatory cytokine actions of IL-6 and other stimulated cells. Upon the release of IL-10, there is a multifold increase in an array of cytokines including INF-gamma whose anti-viral and other characteristics signal an increase of GM CSF as well as CSF and Monocyte Chemoattractant Protein (MCP-1). At 30 hours post administration, there is the up regulation of MCP-1 which stimulates the recruitment of T-cells B-cells, Macrophages and other immune cells to the sites of injury and damage to initiate the removal of damaged tissue and begin actual cellular healing. Since the effect of the up regulation of MCP-1 is that those cells can pass through the blood/brain barrier (BBB) which allows the therapeutic help on many neurological conditions as well. The entire cascade initiated by the invention is over in about 120 hours and the immune system is properly reset leaving no trace of the invention and allowing the body to receive another administration to begin the cascade once again as needed.

BRMs of the invention are nontoxic at therapeutic and prophylactic concentrations showing no cytotoxicity or detectable short-term (days) or long-term (weeks and months) side effects upon administration to patients. BRMs safely prompt the existing immune system to modulate its own cytokine circuitry to affect a positive therapeutic or prophylactic outcome.

BRM compositions of the invention can treat and/or prevent a variety of diseases and disorders, and also provide systematic or palliative treatment (e.g., symptomatic treatments) of a patient undergoing conventional therapy. Diseases and disorders that can be treated and/or prevented include, but are not limited to bacterial infection (e.g., Streptococcus, Staphylococcus, Pseudomonas, Listeria, Mycobacteria, Salmonella, Escherichia coli, Yersinia pestis, Klebsiella, Shigella, Clostridum), viral infection (e.g., Hepatitis B and C, Rubella virus, Herpes Simplex virus, retrovirus, varicella zoster virus, human papilloma virus, parvovirus, HIV), or parasitic infection (e.g., plasmodium, Leishmania, Guardia) infection, inflammation, inflammatory bowel disease, amelioration of an inflammatory condition or symptom, chronic inflammatory disease, multiple sclerosis, chronic pain, Alzheimer's, ALS, Lyme disease, cancer, Type 1 and Type 2 diabetes, auto-immune disorders, chronic fatigue syndrome, Rheumatoid arthritis, myasthenia gravis, Celiac disease—sprue, Systemic lupus erythematosus, psoriasis, and combinations thereof. In addition, BRMs of the invention can also be used to treat various neoplasia including but not limited to prostate cancer, breast cancer, lymphoma, pancreatic cancer, kidney cancer, stomach cancer, lung cancer, cervical cancer, colorectal cancer, melanoma, leukemia, tumors, and metastatic disease.

One embodiment of the invention is directed to BRMs that generate systemic alterations of a mammalian cytokine profile. The alterations generated by a particular BRM are correlated with particular diseases and disorders including auto immune diseases and wound healing. Alterations are rarely static, but represent a cascade of multiple interactions that lead to a beneficial immune response. In addition, the alterations remain for only so long as is necessary addressing the particular condition and, preferably, after which the patient's immune system and cytokine levels return to their pre-exposed levels. The BRMs themselves are not believed to be either bacteriostatic or bactericidal, but beneficially alter the cascade of cytokine interactions. Preferably BRMs are isolated from mammalian serum and also preferably from caprine serum. One BRM of the invention is referred to as NPIS40.

NPIS40 directly modulates both inflammatory and anti-inflammatory cytokines, and is not an antibiotic, a bactericide, or a virucide. Cytokine levels were determined before and over a period of time after administration including Interferon gamma, an antiviral cytokine, IL-6, an inflammatory cytokine, and IL-10. Interferon gamma was found to be upregulated and IL-6 was found to be up-regulated, at first, and later suppressed by release of IL-10. IL-6 is known to help the host resist bacterial challenges, but the direct exogenous administration of too much IL-6 can itself be significantly harmful, even deadly. NPIS40 allows the body to control cytokine levels for protection against disease and/or for restoration of levels after resolution of the particular disease. BRMs of the invention do not create an uncontrolled storm of cytokines, but maintain the mechanisms that exist and provide for the self-control of the immune system.

This sequence of events has a beneficial effect in cancer patients and patients with auto-immune disease. In tests with mice administered NPIS40, a greater percentage were able to resist a lethal bacterial challenge as compared to a control group.

Another embodiment of the invention is directed a BRM compound and suitable pharmaceutical compositions that at the therapeutically effective dose upregulate IgA levels of a patient in need thereof. The cause or causative agent of selective IgA deficiency is not currently known or understood. Unlike other Ig deficiencies, IG replacement therapy is not viable for selective IgA deficiency disorders. Administration of BRM of the invention uporegalate IgA. Another embodiment of the invention is directed to one or more components of isolated and preferably purified NPIS40, NPIS40 synthesize de novo and NPIS40 chemically modified. Preferably the one or more components are peptides, a series of peptides, or peptide complexes. It was surprisingly discovered that components of NPIS40 at masses 1103 and 1131 include fibrinopeptide fragments. A number of these fragments were synthesized and found to have the amino acid sequences YLDYDEVDDNRAKLPLDA (SEQ ID NO. 1) with a tyrosine sulfonation and/or a phosphorylation at the first and/or second tyrosine and derived from fibrinopeptide B; GYLDYDEVDDNRAKLPLDA (SEQ ID NO. 4) with a tyrosine sulfonation and/or a phosphorylation at the first and/or second tyrosine and derived from fibrinopeptide B; GYLDYDEVDDNRAKLPLDAR (SEQ ID NO. 5) with a tyrosine sulfonation and/or a phosphorylation at the first and/or second tyrosine and derived from fibrinopeptide B; a fragment derived from fibrinopeptide A of the sequence FLAEGGGV (SEQ ID NO. 6), a fragment of C3 of fibrinogen beta of the sequence SEETKENERFTV (SEQ ID NO. 7); a fragment of C3 of fibrinogen beta of the sequence SAKFSAEEELEIR (SEQ ID NO 8); a fragment of fibrinogen alpha of the sequence IQTADDSDPVGGEFLAEGGGV (SEQ ID NO. 11), which may be unmodified; and a fragment of fibrinogen alpha of the sequence DEAESIEDLGIKGAHATKTGHA (SEQ ID NO. 9), which may be unmodified.

Another embodiment of the invention comprises methods for isolating and preferably purifying NPIS40. Methods for the manufacture of BRMs of the invention from serum comprise obtaining the serum which may be maintained frozen, preferably minus 20° C. or colder. Thawing the serum to about room temperature or colder, pre-filtration of the serum to remove particles and solid materials that may be present, tangential flow filtration against a salt solution, such as sodium chloride, filtration through 0.2 μm filters and storage of the filtered liquid at from about minus 10° C. to about minus 25° C.

Another embodiment of the invention comprises methods for the treatment or prevention of a mammalian disease or disorder, or symptomology treatment, in a patient comprising administering to said patient an effective amount of NPIS40 of the invention. Preferably the mammalian disease or disorder is a bacterial, viral or parasitic infection, a cancer or other neoplasia, a chronic condition such as chronic pain or malaise, or a combination thereof. In such methods, the patient is preferably a mammal such as a human. The effective amount of the isolated peptides is that amount sufficient to produce an effective serum concentration for treating or preventing the specific disease or disorder and, after resolution of the disorder, returning the immune system to its prior pre-exposure condition.

Another embodiment of the invention comprises BRM compositions containing pharmaceutically acceptable carriers and/or salts. Suitable pharmaceutically acceptable carriers include, but are not limited to, water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxyl methylcellulose, polyvinylpyrrolidone, etc. The pharmaceutical compositions of the invention can also be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like, which do not deleteriously react with the active compounds of the invention. A pharmaceutically acceptable salt includes a basic or acidic group of a compound of the invention. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate salts.

Pharmaceutically acceptable salts also include salts prepared from a compound of the invention having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl, N-ethylamine; diethylamine; triethylamine; mono-, his-, or tris-(2-hydroxy-lower alkyl amines), such as mono-, his-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. Other pharmaceutically acceptable salts are described in the Handbook of Pharmaceutical Salts: Properties, Selection, and Use (P. Heinrich Stahl and C. Wermuth, Eds., Verlag Helvetica Chica Acta, Zurich, Switzerland (2002), which is specifically incorporated by reference).

A BRM of the invention includes compounds that can be converted via a chemical or physiological process (e.g., enzymatic processes and metabolic hydrolysis). Such compounds often offer advantages of increased solubility, or delayed release in an organism. Converted BRMs are also intended to include any covalently bonded carriers, which release the active compound in vivo when such compound is administered to a subject. Converted compounds of an active compound may be prepared by modifying functional groups present in the active compound in such a way that the modifications are cleaved, either in routine manipulation or in vivo, to the parent active compound. Convertible mechanisms of compounds of the invention include compounds wherein a hydroxy, amino or mercapto group is bonded to any group that, when the converted compound of the active compound is administered to a subject, cleaves to form a free hydroxy, free amino or free mercapto group, respectively. Examples of converted compounds include, but are not limited to, acetate, formate and benzoate derivatives of an alcohol or acetamide, formamide and benzamide derivatives of an amine functional group in the active compound and the like.

Subjects that can be treated with BRM compounds of the invention are used interchangeably herein with the term patient, and refer to a mammal, more preferably a primate, still more preferably a human. Mammals include, without limitation, humans, primates, wild animals, rodents, feral animals, farm animals, sports animals, domestic and game animals, and pets. Domestic and game animals include cows, horses, pigs, deer, bison, buffalo, feline species, e.g., domestic cat, canine species, e.g., dog, fox, wolf. Patients and/or subjects include any subset of the foregoing, and can be male or female.

Administration of one or more BRM compounds of the invention preferably contains a therapeutically or prophylactically effective amount of the BRM compound. A therapeutically effective amount is that amount which has a beneficial effect to the subject by alleviating one or more symptoms of the disorder or by simply reducing premature mortality. For example, a beneficial effect may be a decrease in pain on an annual or daily basis, a decrease in duration, frequency or intensity of pain crises, decrease in fatigue or an increased endurance or stamina or walking ability to walk a certain distance without shortness of breath, or increased strength. Preferably, a therapeutic amount is that amount of the BRM compound that stimulates or positively enhances the immune system against a specific disease or disorder.

Prophylactic treatments preferably involve administration of a composition of the invention to a subject having a confirmed or suspected disorder without having any overt symptoms. For example, otherwise healthy patients who have been genetically screened and determined to be at high risk for the future development of a disorder may be administered BRM compositions of the invention prophylactically. Administration can begin at birth and continue, if necessary, for life. Both prophylactic and therapeutic uses are readily acceptable because these compounds are generally safe and non-toxic. A prophylactically effective amount refers to an amount sufficient to affect a beneficial or desired clinical result upon treatment, or an amount of a compound of this invention sufficient to confer a prophylactic effect on the treated subject.

Therapeutically or prophylactically effective amounts will vary, as recognized by those skilled in the art, depending on the specific disease treated, the route of administration, the carrier, excipient or salt form selected, and the possibility of combination therapy. Generally, a therapeutically or prophylactically effective amount can vary with the subject's history, age, condition, sex, as well as the severity and type of the medical condition in the subject, and administration of other pharmaceutically active agents. The effective amount can be empirically determined by those of ordinary skill in the art, and is expected to be from about 0.001 μg of the one or more BRM compounds/ml of total blood volume to 100 μg/ml, preferably between about 0.1 μg/ml and 50 μg/ml, and more preferably between about 1 μg/ml and 10 μg/ml.

Administration can also be by oral, parenteral, sublingual, rectal such as suppository or enteral administration, or by pulmonary absorption or topical application. Parenteral administration may be by intravenous (IV) injection, subcutaneous (s.c.) injection, intramuscular (i.m.) injection, intra-arterial injection, intrathecal (i.t.) injection, intra-peritoneal (i.p.) injection, intraventricular, intracapsular, intraorbital, intracardiac, intradermal, transtracheal, subcuticular, intraarticular, sub capsular, subarachnoid, intraspinal, intracerebro spinal, and intrasternal injection, infusion and other injection or infusion techniques or direct injection or other administration to the subject. Parenteral administration is preferably via intravenous injection or subcutaneous injection. In some embodiments, a composition can be administered by transdermal transfusion such as with a dermal or cutaneous patch, by direct contact with, for example, bone marrow through an incision or some other artificial opening into the body. Compositions may also be administered to the nasal passages as a spray. Arteries of the nasal area provide a rapid and efficient access to the bloodstream and immediate access to the pulmonary system. Access to the gastrointestinal tract, which can also rapidly introduce substances to the blood stream, can be gained using oral, enema, or injectable forms of administration. Compositions may be administered as a bolus injection, spray, inhalant, or administered sequentially over time (episodically) such as every two, four, six or eight hours, every day (QD) or every other day (QOD), or over longer periods of time such as weeks to months. Compositions may also be administered in a timed-release fashion such as by using slow-release resins and other timed or delayed release materials and devices.

Orally active BRM compositions are more preferred as oral administration is often the safest, most convenient and economical mode of drug delivery. But oral administration is disadvantageous because compositions are often poorly absorbed through the gastrointestinal lining. Compounds which are poorly absorbed tend to be highly polar. Consequently, compounds which are effective, as described herein, may be made orally bioavailable by reducing or eliminating their polarity. This can often be accomplished by formulating a composition with a complimentary reagent which neutralizes its polarity, or by modifying the compound with a neutralizing chemical group. Oral bioavailability is also a problem because drugs are exposed to the extremes of gastric pH and gastric enzymes. These problems can be overcome in a similar manner by modifying the molecular structure to withstand very low pH conditions and resist the enzymes of the gastric mucosa such as by neutralizing an ionic group, by covalently bonding an ionic interaction, or by stabilizing or removing a disulfide bond or other relatively labile bond.

Pharmaceutical compositions comprising one or more BRMs suitable for oral administration can be in the form of capsules, tablets, pills, lozenges, cachets, dragees, powders, granules; or as a solution or a suspension in an aqueous or non-aqueous liquid; or as an oil-in-water or water-in-oil liquid emulsion; or as an elixir or syrup; and the like; each containing a predetermined amount of a compound of the present invention as an active ingredient. When intended for oral administration in a solid dosage form (i.e., as capsules, tablets, pills and the like), the pharmaceutical compositions of the invention will typically comprise a compound of the present invention as the active ingredient and one or more pharmaceutically-acceptable carriers, such as sodium citrate or di-calcium phosphate. Optionally or alternatively, such solid dosage forms may also comprise: filters or extenders, such as starches, microcrystalline cellulose, lactose, sucrose, glucose, mannitol, and/or silicic acid; binders, such as carboxy-methylcellulose, alginates, gelatin, polyvinylpyrrolidone, sucrose and/or acacia; humectants, such as glycerol; disintegrating agents, such as agar-agar, calcium carbonate, potato or tapioca starch, alginic acid, certain silicates, and/or sodium carbonate; solution retarding agents, such as paraffin; absorption accelerators, such as quaternary ammonium compounds; wetting agents, such as cetyl alcohol and/or glycerol monostearate; absorbents, such as kaolin and/or bentonite clay; lubricants, such as talc, calcium stearate, magnesium stearate, solid polyethylene glycols, sodium lauryl sulfate, and/or mixtures thereof; coloring agents; and buffering agents.

Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with one or more sweetening or flavoring agents, coloring matter or dyes, and, if so desired, emulsifying and/or suspending agents, together with such diluents as water, ethanol, propylene glycol, glycerin and various like combinations thereof.

Release agents, wetting agents, coating agents, sweetening, flavoring and perfuming agents, preservatives and antioxidants can also be present in the pharmaceutical compositions of the invention. Examples of pharmaceutically-acceptable antioxidants include: water-soluble antioxidants, such as ascorbic acid, cysteine hydrochloride, sodium bisulfate, sodium metabisulfate sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal-chelating agents, such as citric acid, ethylenediamine tetraacetic acid (EDTA), sorbitol, tartaric acid, phosphoric acid, and the like. Coating agents for tablets, capsules, pills and like, include those used for enteric coatings, such as cellulose acetate phthalate (CAP), polyvinyl acetate phthalate (PVAP), hydroxypropyl methylcellulose phthalate, methacrylic acid, methacrylic acid ester copolymers, cellulose acetate trimellitate (CAT), carboxymethyl ethyl cellulose (CMEC), hydroxypropyl methyl cellulose acetate succinate (HPMCAS), and the like.

In addition, the pharmaceutical compositions of the present invention may optionally contain opacifying agents and may be formulated so that they release the active ingredient only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

If desired, pharmaceutical compositions of the present invention may also be formulated to provide slow or controlled release of the active ingredient using, by way of example, hydroxypropyl methyl cellulose in varying proportions; or other polymer matrices, liposomes and/or microspheres. Sustained release compositions can be formulated including those wherein the active component is derivatized with differentially degradable coatings, e.g., by microencapsulation, multiple coatings, etc.

Preferably a pharmaceutical acceptable BRM composition of the invention does not contain sufficient endotoxin to promote a pyrogenic response.

Individual pulses of BRM compositions as disclosed herein can be administered to the patient continuously over a period of several hours, such as about 2, 4, 6, 8, 10, 12, 14 or 16 hours, or several days, such as 2, 3, 4, 5, 6, or 7 days, preferably from about 1 hour to about 24 hours and more preferably from about 3 hours to about 9 hours. Alternatively, periodic doses can be administered in a single bolus or a small number of injections of the composition over a short period of time, typically less than 1 or 2 hours. In certain instances, there are positive consequences that raise the patient's standard of living such as, for example, increased activity or mobility, fewer side-effects, fewer hospital stays or visits to the physician.

The interval between pulses or the interval of no delivery can be greater than 24 hours or can be greater than 48 hours, and can be for even longer such as for 3, 4, 5, 6, 7, 8, 9 or 10 days, two, three or four weeks or even longer. The interval between pulses can be determined by one of ordinary skill in the art. Alternatively, in some embodiments, the interval between pulses can be calculated by administering another dose of a BRM composition when the active component of the composition is no longer detectable in the patient prior to delivery of the next pulse. Alternatively, intervals can also be calculated from the in vivo half-life of the composition. The number of pulses in a single therapeutic regimen can be as little as two, but can be from about 5 to 10, 10 to 20, 15 to 30 or more.

In some embodiments, a subject can receive one or more compositions comprising a BRM composition for life according to the methods of this invention, for example, where the subject has a permanent or incurable disease or disorder. Compositions can be administered by most any means, and can be delivered to the subject as an oral formulation, or injection (e.g. intravenous, subcutaneous, and intra-arterial), infusion or instillation.

In some embodiments, a composition comprising a BRM composition can be administered to a subject before a chemotherapeutic treatment, or radiation treatment is administered to the subject. In alternative embodiments, a BRM composition can be co-administered to a subject with another pharmaceutical composition comprising one or more additional agents.

Another embodiment of the invention comprises an isolated antibody or antibody fragment that is specifically reactive against BRM components of the invention.

Another embodiment of the invention comprises an isolated nucleic acid that encodes the sequences of the peptides of NPIS40 of the invention. Alternatively, nucleic acids may hybridize in whole or in part to nucleic acid that encode such peptides.

Another embodiment of the invention comprises compositions comprising one or more BRMs of the invention as adjuvants for vaccines. These compounds can be administered before, with or after administration to the patient of the particular vaccine. Adjuvants boost the immune system response to a vaccine and are preferably administered to the patient in the same manner as the vaccine such as, for example, as an intra venous, intraperitoneal or intramuscular injection.

Another embodiment of the invention comprises compositions containing one or more compounds of the invention to which are coupled one or more antigenic portions of an antigen. Preferably the antigen is derived from a pathogen that poses a risk of infection to a patient. Coupling of the BRM or modified chemically BRM to an antigen may be via non-covalent bonding, hydrogen bonding or covalent bonding. Coupling is preferably via covalent bonding performed via a coupling agent. Suitable coupling agents include, for example, a cyano activating compound such as, for example, 1-cyano-4-(dimethylamino)-pyridinium tetrafluoroborate (CDAP) or an amino-oxy agent creating a conjugate of one or more BRM compounds, or chemically or structurally modified BRM compounds, coupled with an antigen or antigenic portion of an antigen, preferably of a pathogen. The antigenic portion may be, for example, a protein, a peptide, an oligopeptide, a polysaccharide, a carbohydrate, an organic molecule, a lipid, a fatty acid, a membrane fraction, a specific chemical structure or a combination thereof (e.g., lipopeptide, lipoprotein, organo-fatty acid, haptenated protein, etc.). Conjugate formation via aminoxy chemistry using an aminooxy reactive agent is disclosed and described in U.S. Patent Application Publication No. 2005-0169941 filed Jan. 27, 2005, and U.S. Patent Application Publication No. 2013-0302877 filed Jun. 12, 2013 (specifically incorporated by reference). Conjugation chemistry with a CDAP activating agent is disclosed and described in U.S. Pat. No. 5,849,301 and U.S. Pat. No. 6,299,881 (specifically incorporated by reference).

The vaccines that may be prepared in accordance with the invention include bacterial, viral and parasitic vaccines such as, for example, Diphtheria vaccine; Pertussis (subunit) vaccine; Tetanus vaccine; H. influenzae type b (polyribose phosphate); S. pneumoniae, all serotypes; E. coli, endotoxin or J5 antigen (LPS, Lipid A, and Gentabiose); E. coli, O polysaccharides (serotype specific); Klebsiella, polysaccharides (serotype specific); S. aureus, types 5 and 8 (serotype specific and common protective antigens); S. epidermidis, serotype polysaccharide I, II, and III (and common protective antigens); N. meningitidis, serotype specific or protein antigens; Polio vaccine; Mumps, measles, rubella vaccine; Respiratory Syncytial Virus; Rabies; Dengue vaccine, Yellow Fever vaccine, Zika vaccine, Hepatitis A, B, C, and others; Human Immunodeficiency Virus I and II (GP120, GP41, GP160, p24, others); Herpes Simplex Virus types 1 and 2; CMV (cytomegalovirus); EBV (Epstein-Barr virus); Varicella/Zoster; Malaria; Tuberculosis; Candida albicans, other candida; Pneumocystis carinii; Mycoplasma; Influenzae viruses A and B; Adenovirus; Group A streptococcus, Group B streptococcus, serotypes, Ia, Ib, II, and III; Pseudomonas aeroginosa (serotype specific); Rhinovirus; Parainfluenzae (types 1, 2, and 3); Coronaviruses; Salmonella; Shigella; Rotavirus; Enteroviruses; Chlamydia trachomatis and pneumoniae (TWAR); and Cryptococcus neoformans. Preferably vaccines of the invention prevent bacterial, viral and parasitic infections such as infections caused by Mycobacterium tuberculosis, Plasmodium falciparum,

The following examples illustrate embodiments of the invention, but should not be viewed as limiting the scope of the invention.

### EXAMPLES

### Example 1

### Serum Collection and Purification of BRM

Sterile serum was collected from sheep certified free of any known disease, and sterile filtered. Each serum lot shipped was certified sterile and free of Mycoplasma species. All production of serum was carried out in accordance with cGMP guidelines with further processing.

Sterile goat serum (South Pacific Sera; Christchurch New Zealand) was processed in accordance with ISO9000 quality system under the rules of GMP (Good Manufacturing Practice). Serum plus a saline solution were passed through a 10kDa membrane followed by viral filtration with 0.2 μm filter into a flexible bioprocess container before being frozen or dispensed into 2 ml injection bottles containing 1.5 ml of liquid each. BCA protein assay (spectrophotometric scan) covering the range 190 nm to 340 nm with a protein determination by the Waddell Method. Frozen goat serum was placed at ambient temperature to partially thaw. Pooled partially thawed serum was tested for bio burden. Thawed serum was filtered and rinsed with 500 ml of a 0.5 percent NaCl solution. Sample was tested to ensure no endotoxin was present. A tangential flow system was established using two Pellicon 3 Regenerated Cellulose Mini Cassettes. Cassettes were pre conditioned by re-circulating with 0.5% NaCl prior to diafiltration. Serum was filtered with a retentate flow of approximately 0.92 L/min and a feed pressure of approximately 1.9 Bar. Samples of permeate were obtained every 5 to 10 minutes for spectrophotometric scanning. When consecutive scans appear similar, the diafiltration process was started. Serum was stirred and simultaneously pumped with 0.5 percent NaCl into containers. The filtration process was continued until the filtrate volume was twice that of the initial volume of pre filtered (Raw) goat serum. 10 ml samples were obtained, labeled and stored at minus 10° C. to minus 25° C.

Viral Filtration: Using a calibrated pressure gauge in line with a peristaltic pump and the Millipore Viral filter pre rinse the same with approximately 75 L of 0.5% NaCl. The processed remaining diffusate was filtered. Liquid that passed through the tangential flow filtration) into the 40 L vessel at a pressure not exceeding 5.5 Bar was recorded including details in the MBR, start and finish time, and initial and final pressure together with average flow rate. The vessel was weighed and the filtrate volume determined assuming 1 Kg equates to I Litre recording the same in the MBR. Sample at 4×10 mls were labeled and stored at 2° C. to 8° C. testing again for bio burden and endotoxin. An integrity test of the Viral Filter was determined.

Filtration and Filling: A calibrated pressure gauge was connected in line of a peristaltic pump and a Pall Kleenpak, Fluorodyne II filter. The assembly was connected to a Hyclone 5 L flexible bag and approximately 5 Litres filtered. Pressure should not exceed 3 Bar or material will become compromised and may need replacement. The amount of filtrate was recorded in each bag with four 10 ml samples labelling and retained from the process and recorded in the MBR. One sample was retained for endotoxin and bio burden testing. The remainder of product was stored at minus10° C. to minus 25° C. The final product retentate was stored at minus 4° C. for 24 hours prior to vial filling. Product produced under Option 2 was referred to as NPIS40.

### Example 2

### Oligosaccharide and Oligonucleotide Analysis of NPIS40

NPIS40 collected according to Example 1 was first characterized by lectin-based enrichment followed by HILIC LC-MS for the presence of oligosaccharide and nanospray infusion in negative mode for oligonucleotides. Neither assay generated a response above background. If either sort of oligo is present, it was either below the limits of detection of not observable using these methods. In addition, no evidence of the presence of glycans was observed.

### Example 3

### Mass Spectrometer Analysis

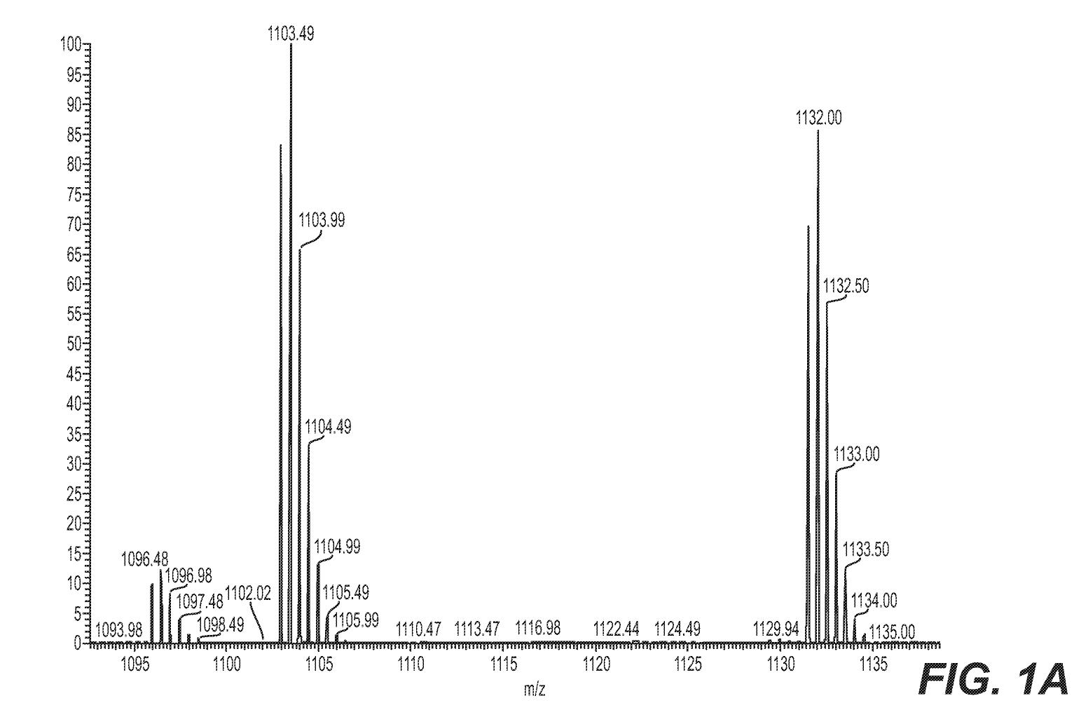

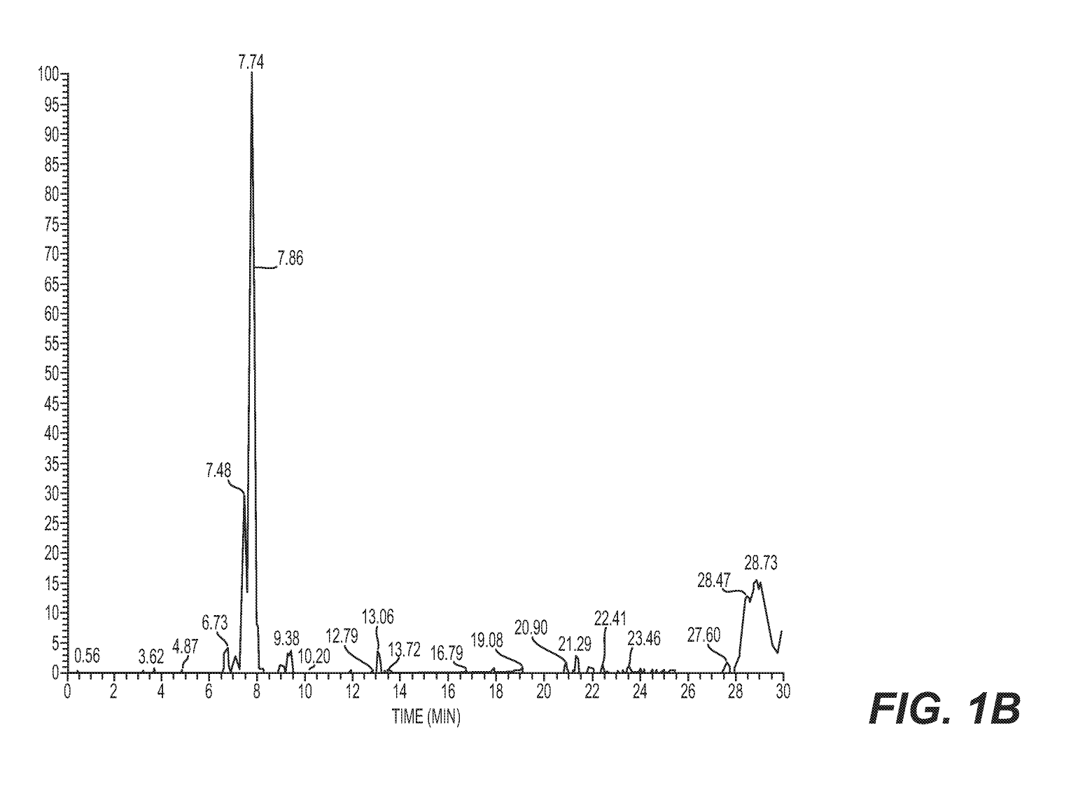

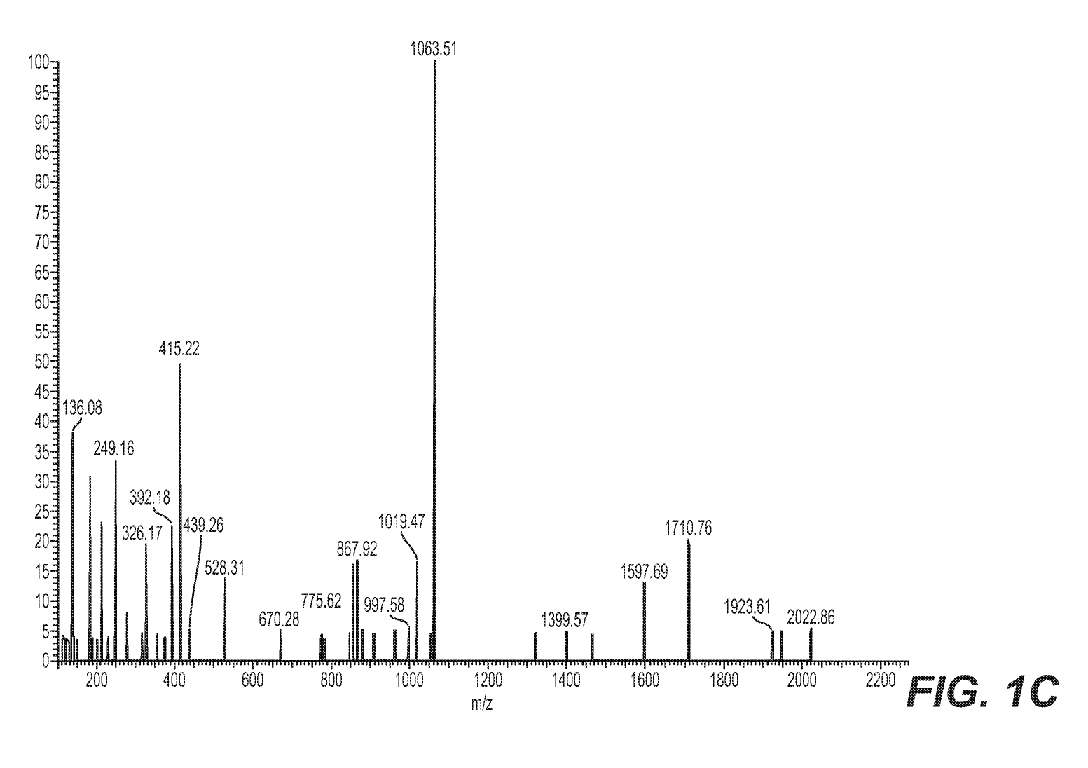

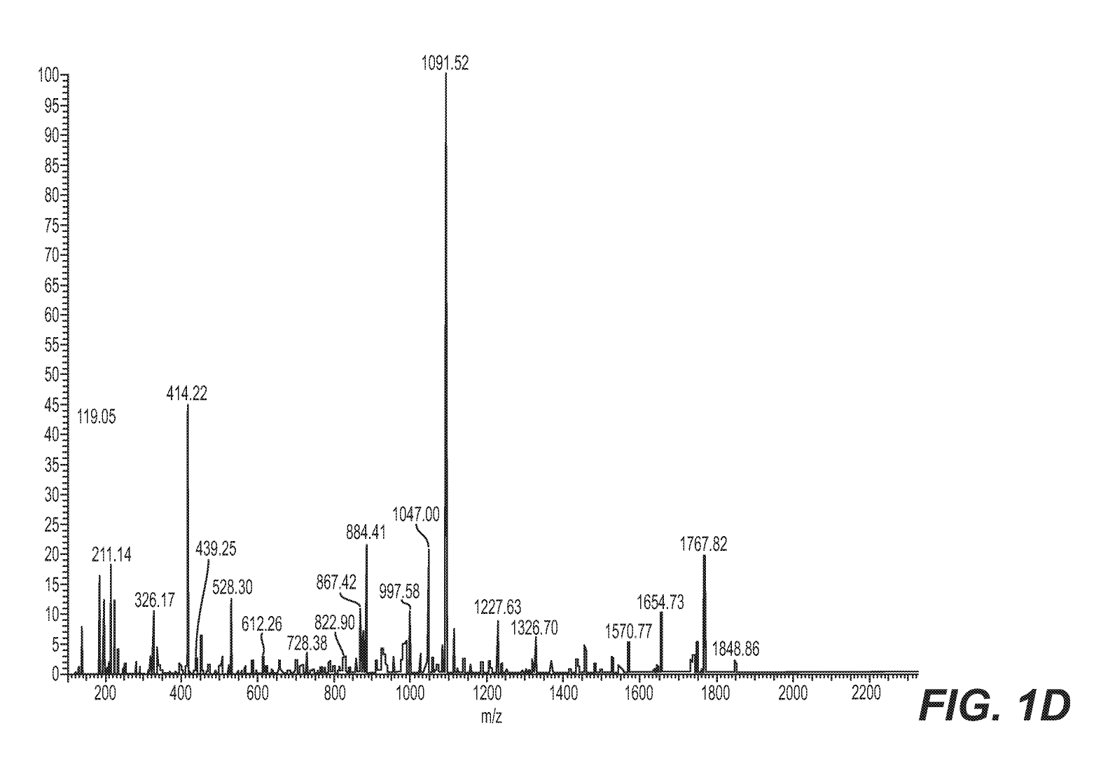

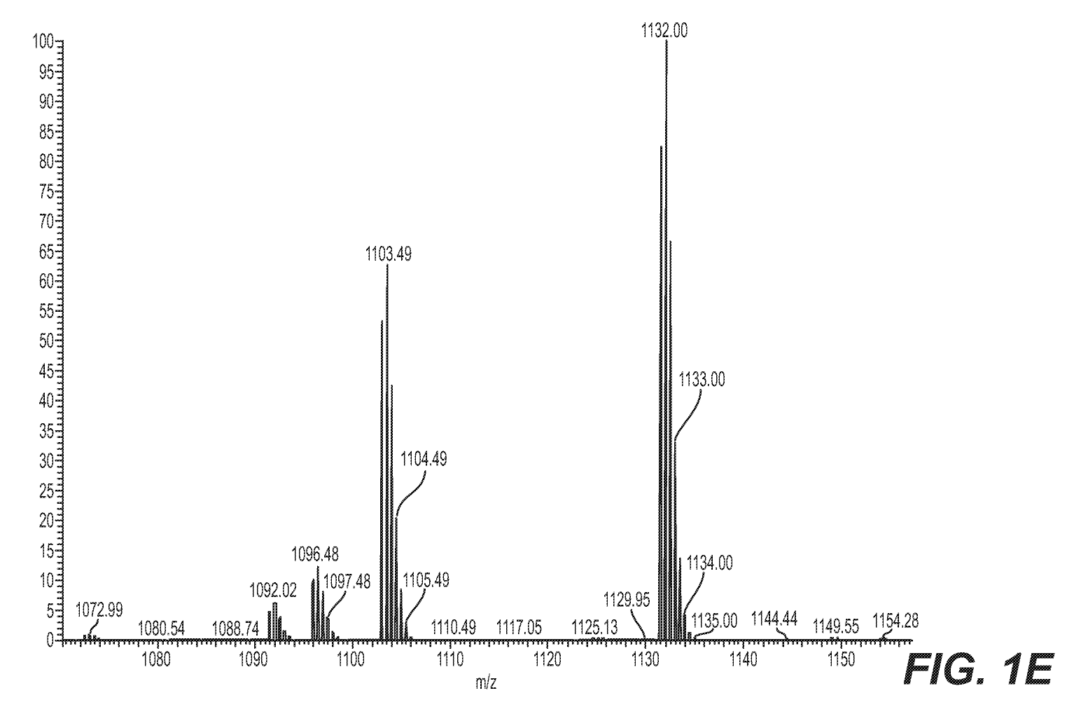

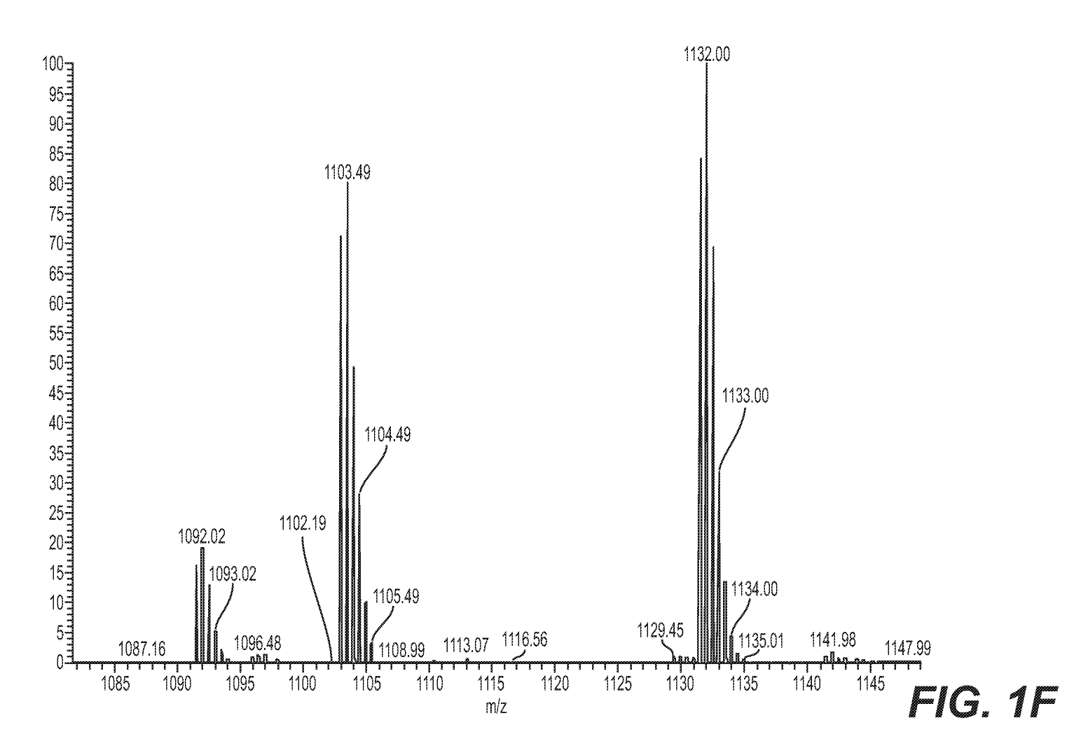

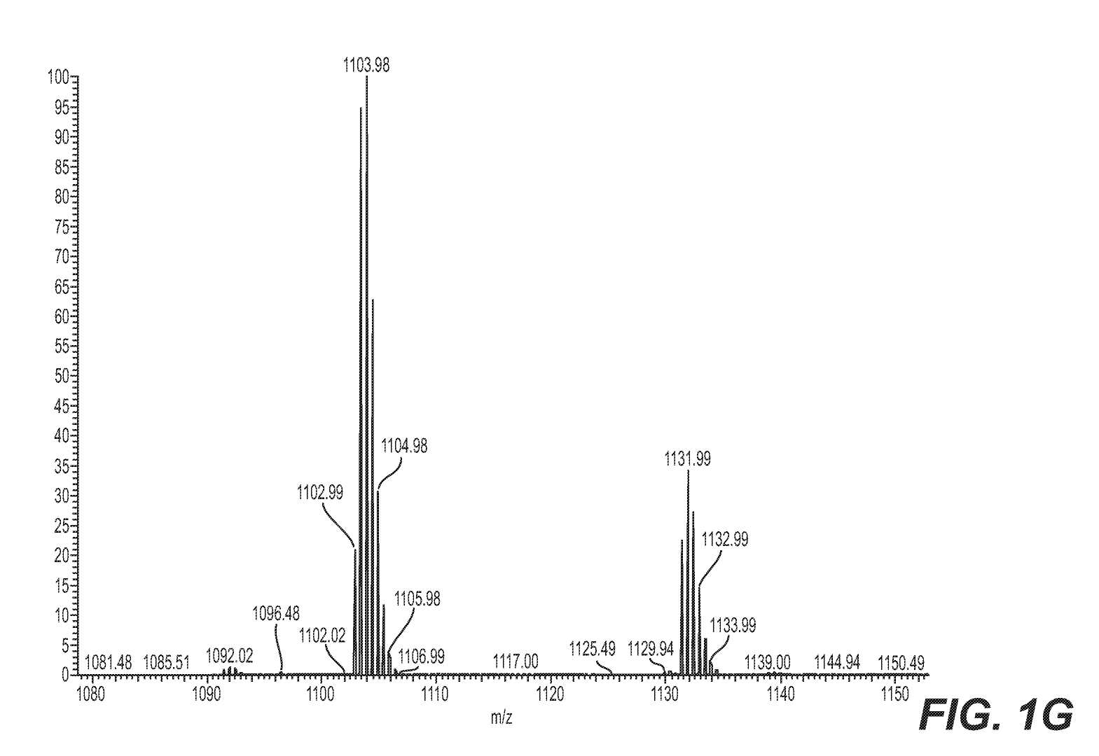

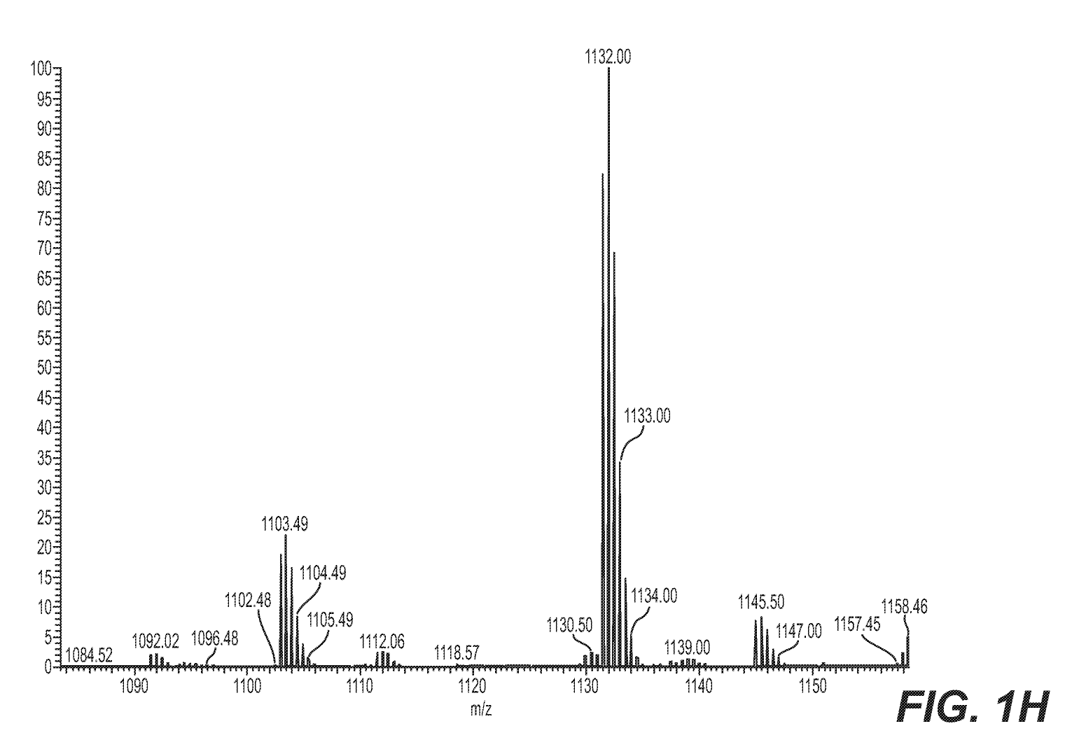

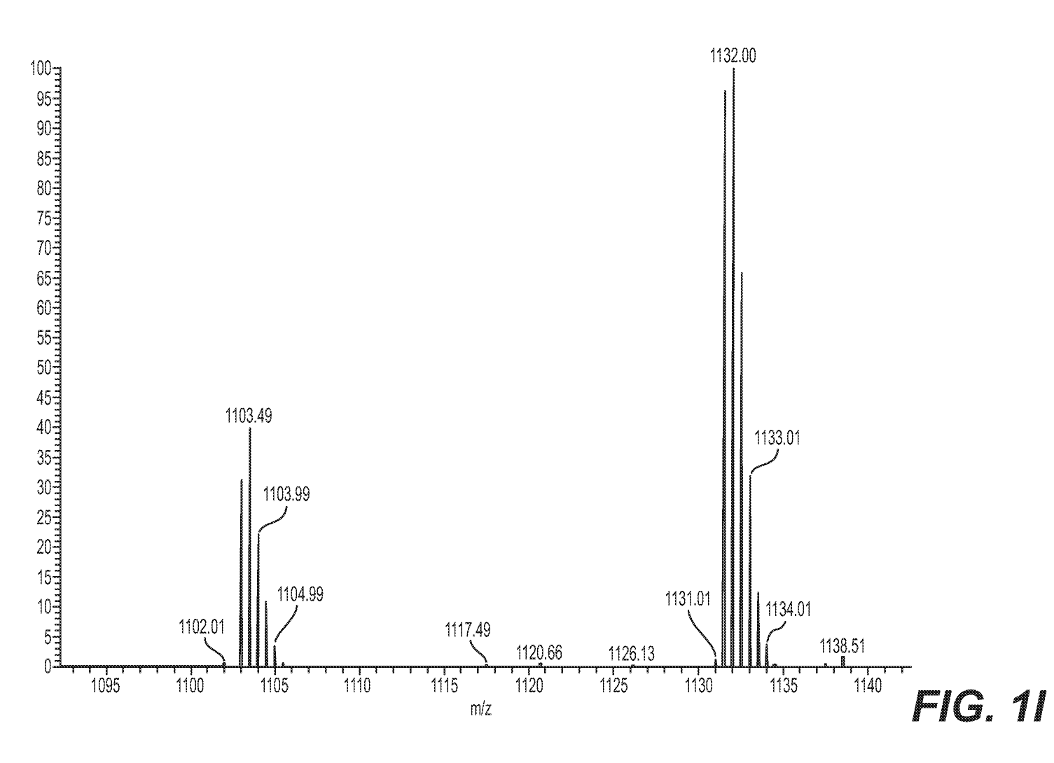

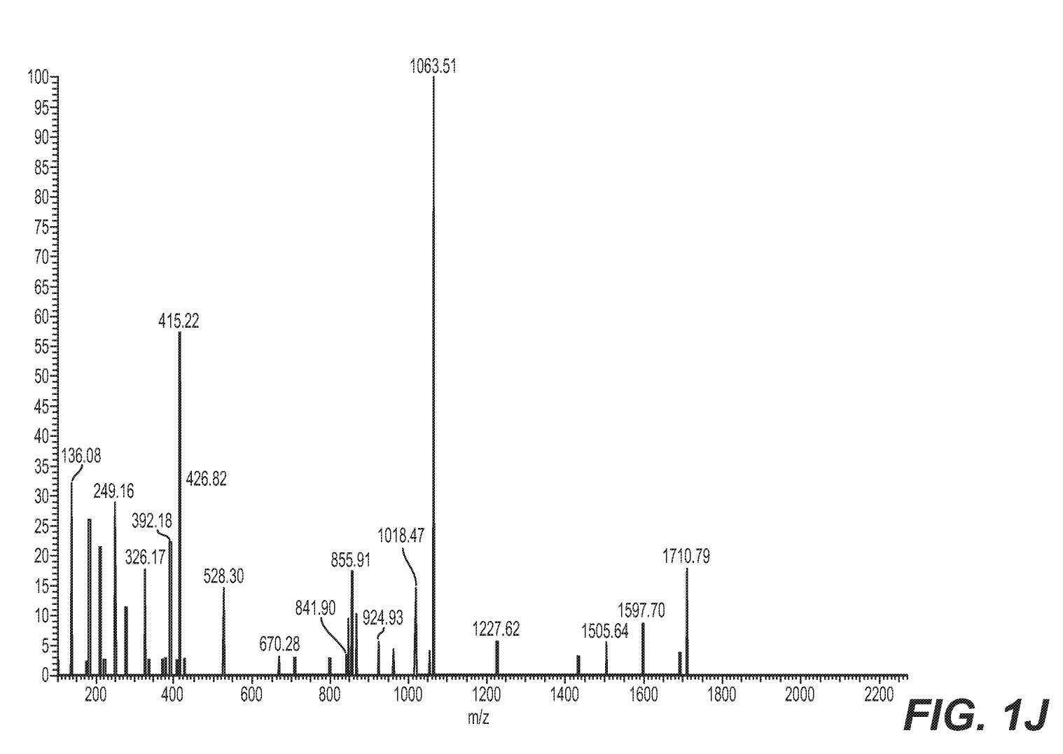

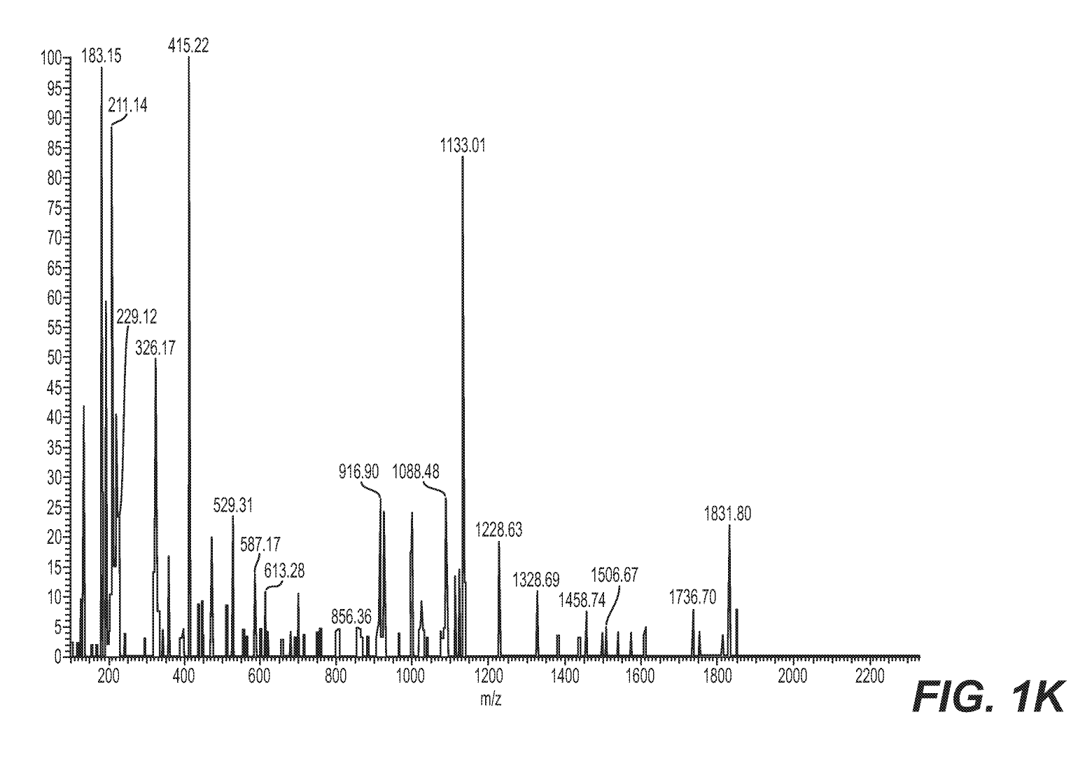

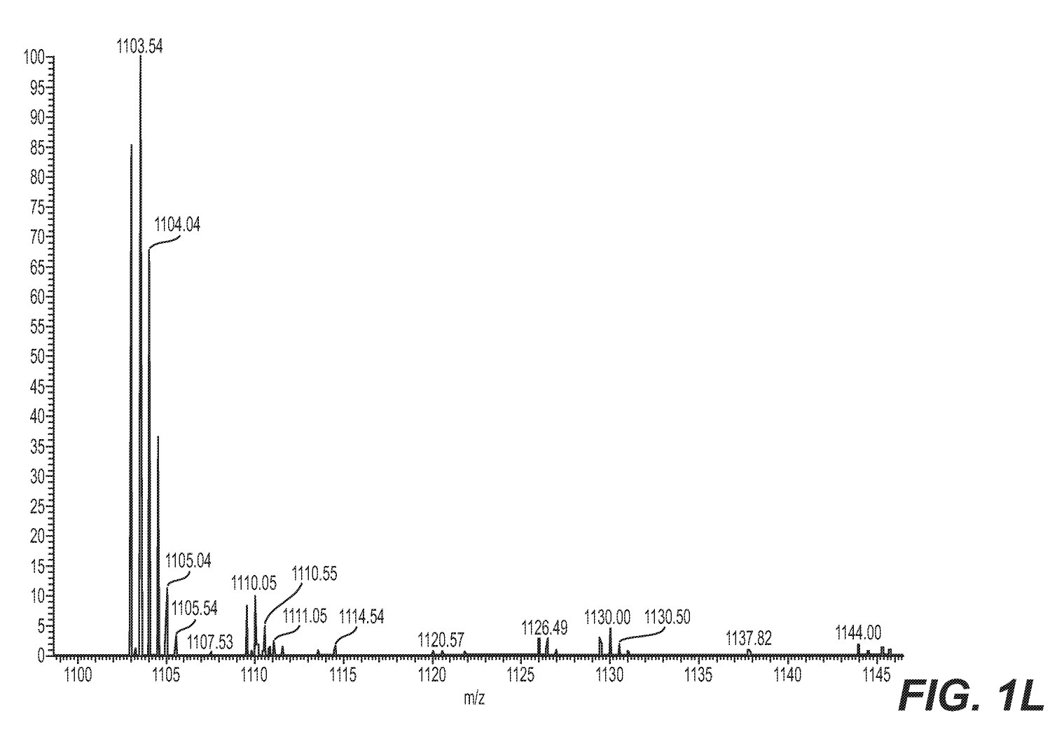

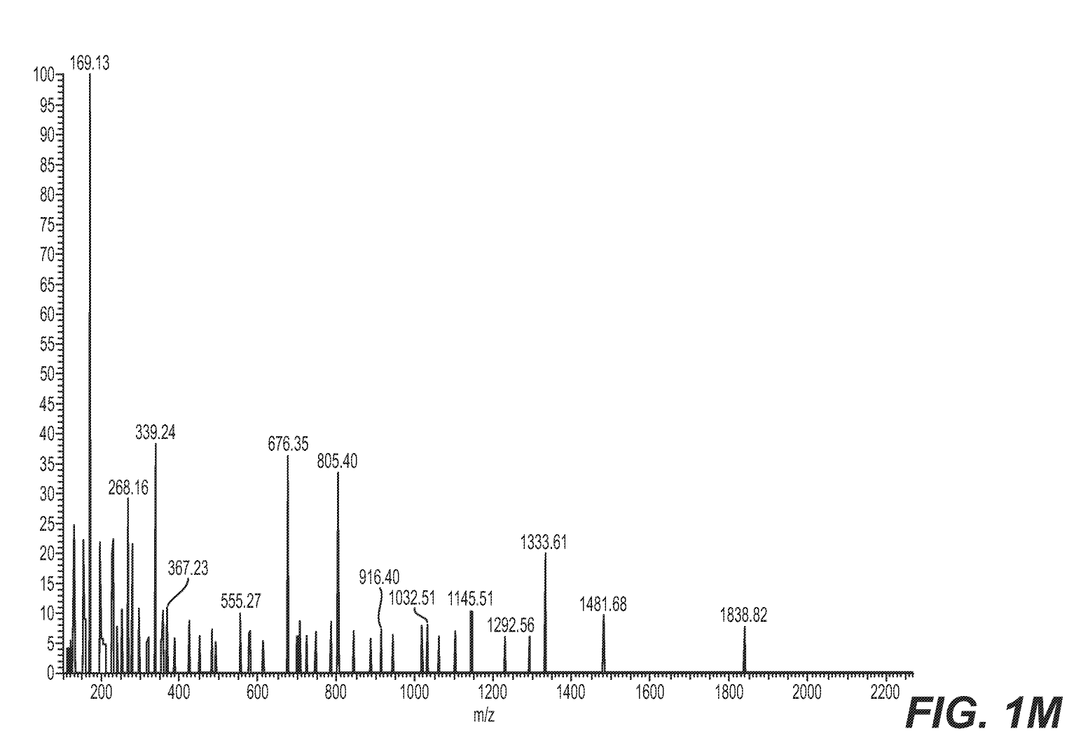

Seven samples of serum collected according to Example 1 were prepared by filtering isolated serum through a 30 kDa MWCO (molecular weight cut-off filter) filter, followed by solid-phase extraction and vacuum concentration, named as (1) de novo synthesized product sample, (2) GT150909-Sec, (3) GT150930-1, (4) GT150904, (5) SF70507-6, (6) caprine ICPF, and (7) human ICPF (see FIGS. 1A-1M).

UPLC-MS/MS was performed using Easy-LC 1000 coupled to a Q-Exactive mass spectrometer. The column was a 25 cm by 200 micron PepSwift monolith. Intact (full MS) and fragmentation (MS/MS) spectra were acquired.

Samples 1-6 are composed primarily of two compounds, one at m/z 1102.9895 and one at 1131.5001, corresponding to masses of 2203.9632 and 2260.9844. The mass difference between the compounds is 57.0212. This mass difference observed may be attributed to an iodoacetamide derivative or to several amino acid substitutions. Fragmentation spectra indicate that there is significant overlap between samples and none have been found in caprine libraries. Sample 1 has the same compound at m/z 1102.9, as determined by fragmentation spectra, the compound at m/z 1131.5, despite having the exact same parent mass, has a slightly different fragmentation spectrum. An explanation for this observation is that the amino acid composition is the same but the sequence order is different at one or more positions. These two compounds are not observed in sample 7. The components identified in each sample are set forth in Table 1.

TABLE 1
Sample 1 de novo synthesized product sample
supplied as a pill
Fibrinopeptide B fragment, modified
GYLDYDEVDDNRAKLPLDA (SEQ ID NO. 4) with sulfation
at the 1st tyrosine
Fibrinopeptide B fragment
GYLDYDEVDDNRAKLPLDA (SEQ ID NO. 4)
Fibrinopeptide A fragment
FLAEGGGV (SEQ ID NO. 6)
Fibrinogen chain alpha fragment
LCLVLSLVGAIQTADDSDPVGGEFLAEGGGV (SEQ ID NO. 12)
Plus minor components:
gi|162424563|gb|ABX89978.1|immunoglobulin mu heavy
chain constant region, partial [Capra hircus]
gi|803080809|ref|XP_012034584.1|PREDICTED:
complement C3 isoform X1, partial [Ovis aries]
gi|426230302|ref|XP_004009215.1|PREDICTED:
sodium/iodide cotransporter isoform X1 [Ovis aries]
gi|803322707|ref|XP_012019563.1|PREDICTED: kin of
IRRE-like protein 1 isoform X2 [Ovis aries musimon]
gi|426254655|ref|XP_004020992.1|PREDICTED: nuclear
transcriptional regulator 1-like protein
[Ovis aries]
Sample 2 GT150909-Sec
Fibrinopeptide B fragment, modified
GYLDYDEVDDNRAKLPLDA (SEQ ID NO. 4) with sulfation
at the 1st tyrosine
Fibrinopeptide B fragment
YLDYDEVDDNRAKLPLDA (SEQ ID NO. 1)
Plus minor components:
gi|803058249|ref|XP_012031490.1|PREDICTED: myosin-9
isoform X1 [Ovis aries]
gi|926707690|ref|XP_013824344.1|PREDICTED: short
palate, lung and nasal epithelium carcinoma-
associated protein 2B-like [Capra hircus]
Sample 3 GT150930-1.
Fibrinopeptide B fragment, modified
GYLDYDEVDDNRAKLPLDA (SEQ ID NO. 4) with sulfation
at the 1st tyrosine
Fibrinopeptide B fragment, modified
YLDYDEVDDNRAKLPLDA (SEQ ID NO. 1) with sulfation
at the 1st tyrosine
Fibrinopeptide B fragment
GYLDYDEVDDNRAKLPLDA (SEQ ID NO. 4)
Sample 4 GT150904.
Fibrinopeptide B fragment, modified
GYLDYDEVDDNRAKLPLDA (SEQ ID NO. 4) with sulfation
at the 1st tyrosine
Fibrinopeptide B fragment, modified
GYLDYDEVDDNRAKLPLD (SEQ ID NO. 3) with sulfation
at the 1st tyrosine
Fibrinopeptide B fragment, modified
YLDYDEVDDNRAKLPLDA (SEQ ID NO. 1) with sulfation
at the 1st tyrosine
Plus minor components:
gi|548486143|ref|XP_005686787.1|PREDICTED:
thymosin beta-10 [Capra hircus]
gi|926730042|ref|XP_005701550.2|PREDICTED:
complement C3 [Capra hircus]
gi|926730881|ref|XP_005702023.2|PREDICTED:
complement C4-like [Capra hircus]
gi|803033633|ref|XP_012019984.1|PREDICTED: rho-
associated protein kinase 2 isoform X1 [Ovis aries]
gi|926695622|ref|XP_013820690.1|PREDICTED:
complement C3, partial [Capra hircus]
gi|803262238|ref|XP_011990819.1|PREDICTED:
5′-AMP-activated protein kinase subunit
gamma-2 isoform X5 [Ovis aries musimon]
gi|803230738|ref|XP_011975608.1|PREDICTED:
caldesmon isoform X3 [Ovis aries musimon]
gi|803058071|ref|XP_004007547.2|PREDICTED:
olfactory receptor 10C1-like [Ovis aries]
gi|803317538|ref|XP_012016989.1|PREDICTED: zyxin
isoform X2 [Ovis aries musimon]
Sample 5 SF70507-6
Fibrinopeptide B fragment, modified
GYLDYDEVDDNRAKLPLDA (SEQ ID NO. 4) with sulfation
at the 1st tyrosine
Fibrinopeptide B fragment
GYLDYDEVDDNRAKLPLDA (SEQ ID NO. 4)
Fibrinogen chain alpha fragment
DEAESIEDLGIKGAHATKTGHAKA (SEQ ID NO. 10)
Plus minor components:
gi|803170940|ref|XP_004017232.2|PREDICTED:
fibrinogen alpha chain isoform X1 [Ovis aries]
gi|803080809|ref|XP_012034584.1|PREDICTED:
complement C3 isoform X1, partial [Ovis aries]
Sample 6 Caprine ICPF
Fibrinopeptide B fragment, modified
GYLDYDEVDDNRAKLPLDA (SEQ ID NO. 4) with sulfation
at the 1st tyrosine
Fibrinopeptide B fragment
YLDYDEVDDNRAKLPLDA (SEQ ID NO. 1)
Plus other minor components:
gi|803170940|ref|XP_004017232.2|PREDICTED:
fibrinogen alpha chain isoform X1 [Ovis aries]
gi|926707690|ref|XP_013824344.1|PREDICTED: short
palate, lung and nasal epithelium carcinoma-
associated protein 2B-like [Capra hircus]
gi|685425595|pdb|4LUF|AChain A, Crystal Structure
Of Ovine Serum Albumin
gi|685425596|pdb|4LUH|AChain A, Complex Of Ovine
Serum Albumin With 3,5-diiodosalicylic Acid
gi|926712046|ref|XP_005691214.2|PREDICTED:
fibrinogen alpha chain [Capra hircus]
gi|926710444|ref|XP_013825345.1|PREDICTED: LOW
QUALITY PROTEIN: prothrombin [Capra hircus]
gi|803293300|ref|XP_012006313.1|PREDICTED:
fibrinogen alpha chain isoform X2 [Ovis aries
musimon]
gi|209447321|pdb|2RI4|BChain B, Crystal Structure
Determination Of Goat Methemoglobin At 2.7 Angstrom
gi|426250945|ref|XP_004019193.1|PREDICTED:
coagulation factor XIII A chain [Ovis aries]
gi|926704654|ref|XP_013823271.1|PREDICTED:
galectin-related protein [Capra hircus]
gi|803247887|ref|XP_011983922.1|PREDICTED:
coagulation factor XIII A chain [Ovis aries
musimon]
gi|193085052|gb|ACF10391.1|albumin precursor,
partial [Capra hircus]
gi|803189673|ref|XP_011956845.1|PREDICTED:
complement C4-A-like [Ovis aries]
gi|926730803|ref|XP_013832694.1|PREDICTED:
complement C4-A-like [Capra hircus]
gi|266618518|pdb|3EU1|BChain B, Crystal Structure
Determination Of Goat Hemoglobin (Capra Hircus) At
3 Angstrom Resolution
gi|926715543|ref|XP_013827208.1|PREDICTED: alpha-
1B-glycoprotein [Capra hircus]
gi|803242149|ref|XP_011981169.1|PREDICTED:
galectin-related protein isoform X2 [Ovis aries
musimon]
gi|426224376|ref|XP_004006347.1|PREDICTED: proline-
rich protein 13 [Ovis aries]
gi|237640463|pdb|3D1A|DChain D, Crystal Structure
Determination Of Goat Hemoglobin At 2.61 Angstrom
Resolution
gi|803189675|ref|XP_011956846.1|PREDICTED:
complement C4-A isoform X1 [Ovis aries]
gi|926695372|ref|XP_013820599.1|PREDICTED:
complement C3 isoform X2 [Capra hircus]
gi|548517042|ref|XP_005696356.1|PREDICTED: trem-
like transcript 1 protein [Capra hircus]
gi|926730881|ref|XP_005702023.2|PREDICTED:
complement C4-like [Capra hircus]
gi|209447327|pdb|2RI4|LChain L, Crystal Structure
Determination Of Goat Methemoglobin At 2.7 Angstrom
gi|926729006|ref|XP_013831983.1|PREDICTED: E3
ubiquitin-protein ligase HUWEl isoform X2 [Capra
hircus]
gi|186886474|gb|ACC93613.1|SLC25A38 [Ovis aries]
gi|926721902|ref|XP_013829453.1|PREDICTED:
complement C4 [Capra hircus]
gi|926712071|ref|XP_013825932.1|PREDICTED:
transmembrane protein 131-like [Capra hircus]
gi|426239135|ref|XP_004013482.1|PREDICTED: LOW
QUALITY PROTEIN: sodium channel protein type 4
subunit alpha isoform X1 [Ovis aries]
gi|803217947|ref|XP_011969334.1|PREDICTED: zinc
finger protein 318 isoform X2 [Ovis aries musimon]
gi|803320372|ref|XP_012018397.1|PREDICTED: E3
ubiquitin-protein ligase DTX3L isoform X2 [Ovis
aries musimon]
gi|802980807|ref|XP_012013620.1|PREDICTED: FGGY
carbohydrate kinase domain-containing protein
isoform X3 [Ovis aries]
gi|803244704|ref|XP_011982374.1|PREDICTED: steryl-
sulfatase isoform X2 [Ovis aries musimon]
gi|548506471|ref|XP_005693115.1|PREDICTED: LOW
QUALITY PROTEIN: zinc finger and SCAN domain-
containing protein 4 [Capra hircus]
gi|803284013|ref|XP_012001677.1|PREDICTED:
neuroblast differentiation-associated protein
AHNAK isoform X8 [Ovis aries musimon]
gi|803212895|ref|XP_011966845.1|PREDICTED:
coagulation factor V isoform X2 [Ovis aries
musimon]
gi|803245586|ref|XP_011982785.1|PREDICTED: epsin-2
isoform X5 [Ovis aries musimon]
gi|803321346|ref|XP_012018889.1|PREDICTED:
complement C4-like isoform X2 [Ovis aries musimon]
gi|803240767|ref|XP_011980480.1|PREDICTED: nidogen-
1 isoform X2 [Ovis aries musimon]
Sample 7 Human ICPF
Fibinopeptides and fibrinogen protein fragments of
human or goat were not identified in this sample.
Samples 2, 3 and 5 were the more pure, with the
fewest minor identifiable components.

### Example 4

### Analysis of NPIS40 Peptides

A portion of NPIS40 collected according to Example 1 was analyzed for peptide content. The major components are modified forms of GYLDYDEVDDNRAKLPLDA (SEQ ID NO. 4) and YLDYDEVDDNRAKLPLDA (SEQ ID NO. 1). The modification is on either the first or second tyrosine. The modification may be a sulfation or a phosphorylation. The mass difference between a sulfation and a phosphorylation is small, 0.01 Da. The modification is likely phosphorylation (HPO₃) rather than sulfation (SO₃). And it is more likely on the second Tyr rather than the first. It is also possible that there is a mix at either position and of either modification, although both positions being modified at the same time has not been observed. There are also two fragments of C3 present at about 0.1% of the fibrinogen beta components—SEETKENERFTV (SEQ ID NO. 7) and SAKFSAEEELEIR (SEQ ID NO. 8), and two fragments from fibrinogen alpha at about 0.01% -IQTADDSDPVGGEFLAEGGGV (SEQ ID NO. 11) and DEAESIEDLGIKGAHATKTGHA (SEQ ID NO. 9). All sequences identified were unmodified.

### Example 5

### Mouse Model

NPIS40, collected according to Example 1, or PBS (phosphate buffered saline) was injected i.p. into three of six Swiss Webster mice two days after challenge with a terminal dose of Salmonella (5,000 cfu) along with a plasmid construct with a lux-gene insertion (glows in the dark bacteria). 48 hours after administration of NPIS40, all three mice receiving PBS showed massive infection. Only one of the three mice receiving NPIS40 showed signs of infection and substantially reduced compared to PBS controls.

### Example 6

### Administration of NPIS40

NPIS40 has a profound and immense potential, not only as a therapeutic agent, but also as a prophylactic agent against bacterial pathogens and viral disease agents as well as cell metastasis in neoplastic diseases. Other areas of impact from this work include: NPIS40 can replace use of antibiotics as the therapeutic of choice for infectious diseases, thus reducing usage of antibiotics and decreasing pressure that drives the development of antibiotic resistant bacterial strains. NPIS40 can be a new and affordable therapy for serious infectious diseases such as cholera, dysentery, and tuberculosis that are ravaging the populations of the world's developing nations. NPIS40 can be used as a prophylactic for short term protection in a bio-terrorism arena or other contagion environs. NPIS40 can be administered as a prophylactic to patients undergoing major surgery to prevent post-operative infection. NPIS40 can provide long-term protection as a vaccine adjuvant. NPIS40 can be used as a safe and effective therapeutic for the prevention and treatment of seasonal influenza outbreaks. NPIS40 can be used adjunctively with other therapeutic agents for the treatment of drug resistant infections. NPIS40 can be used to “scavenge” metastatic cells in patients undergoing chemotherapy or surgery for the treatment of neoplastic diseases.

NPIS40 purified according to Example 1 was administered via i.v. injection to a number of patients. Each patient received an initial injection on day 0, followed by repeat injections at about weekly intervals for a total of four injections.

### Example 7

### Analysis of Blood Profiles After Treatment

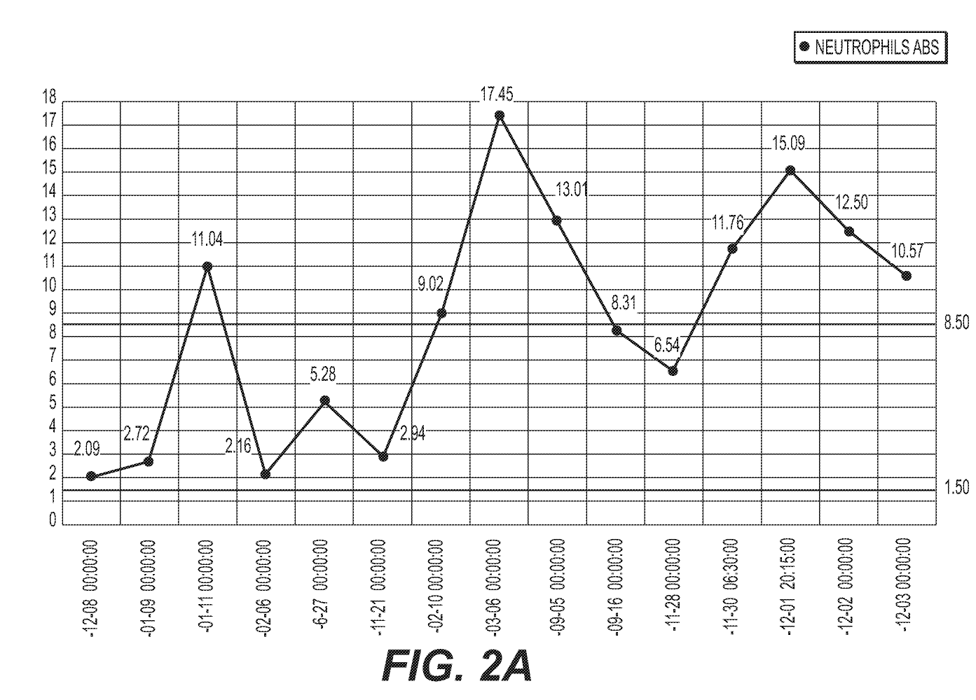

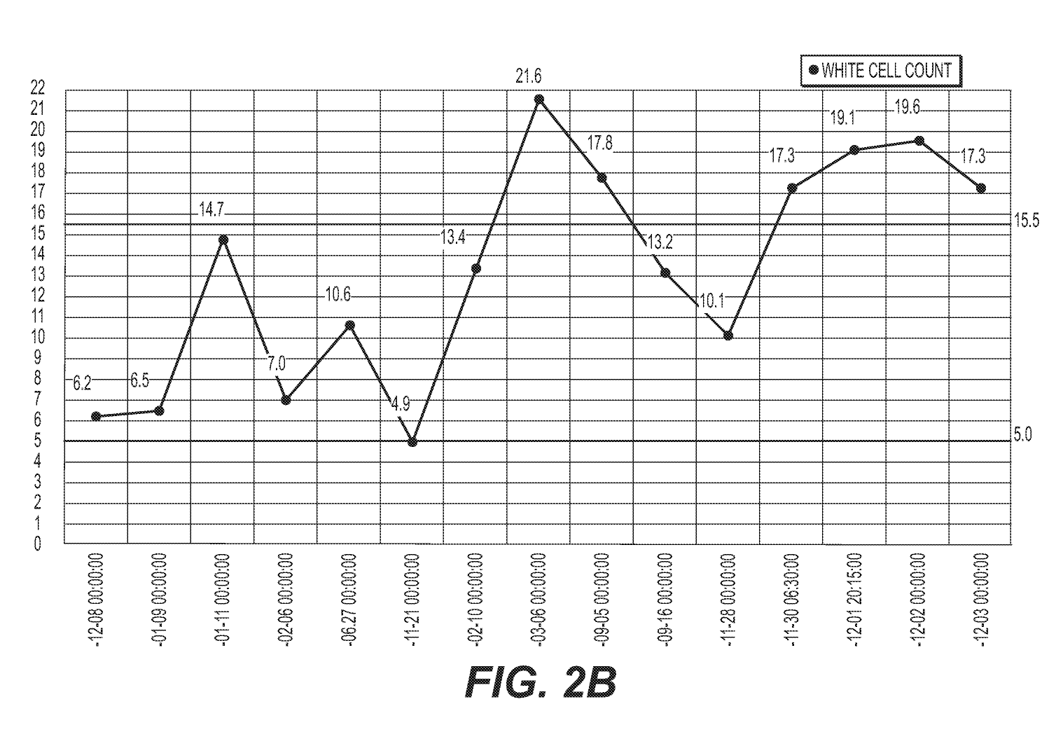

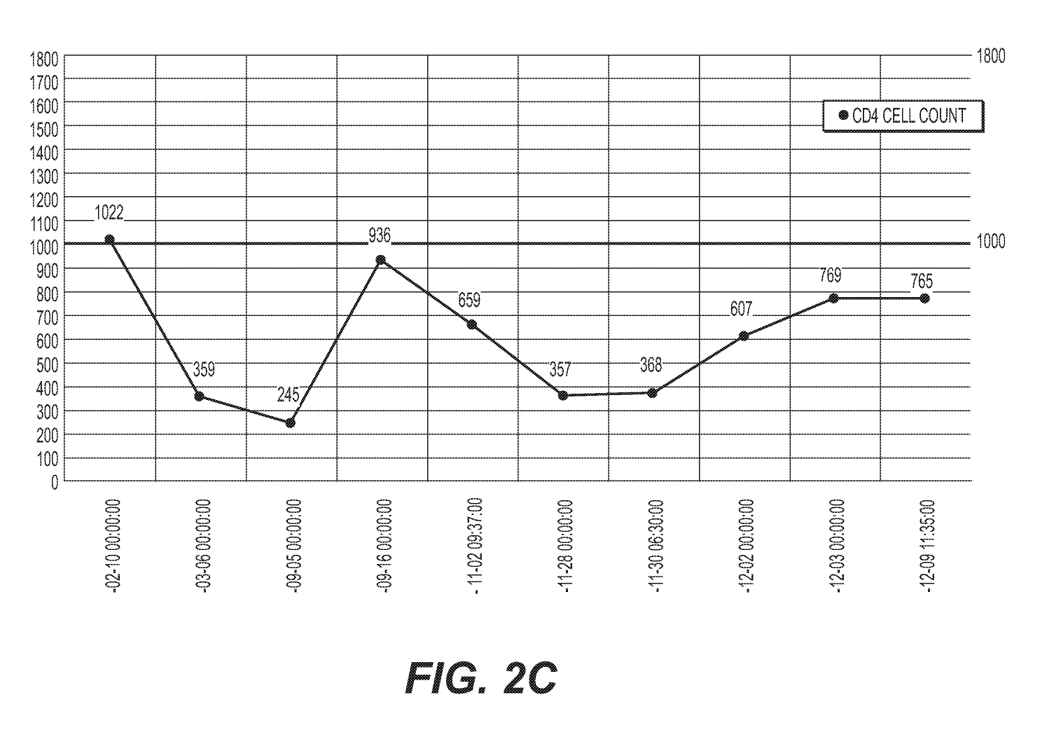

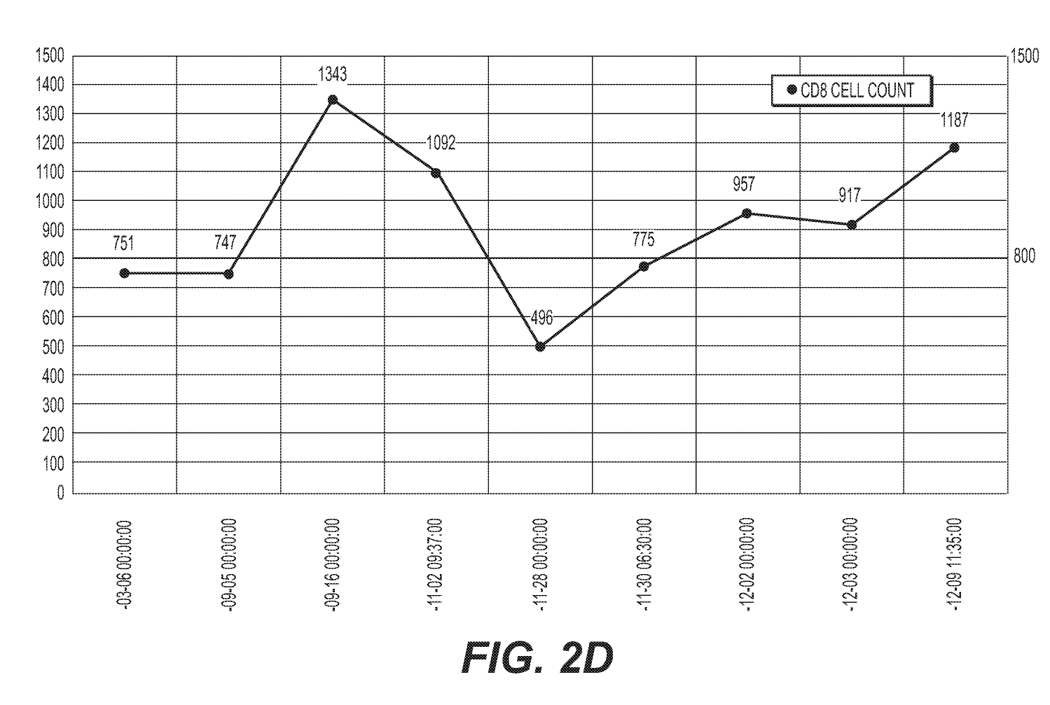

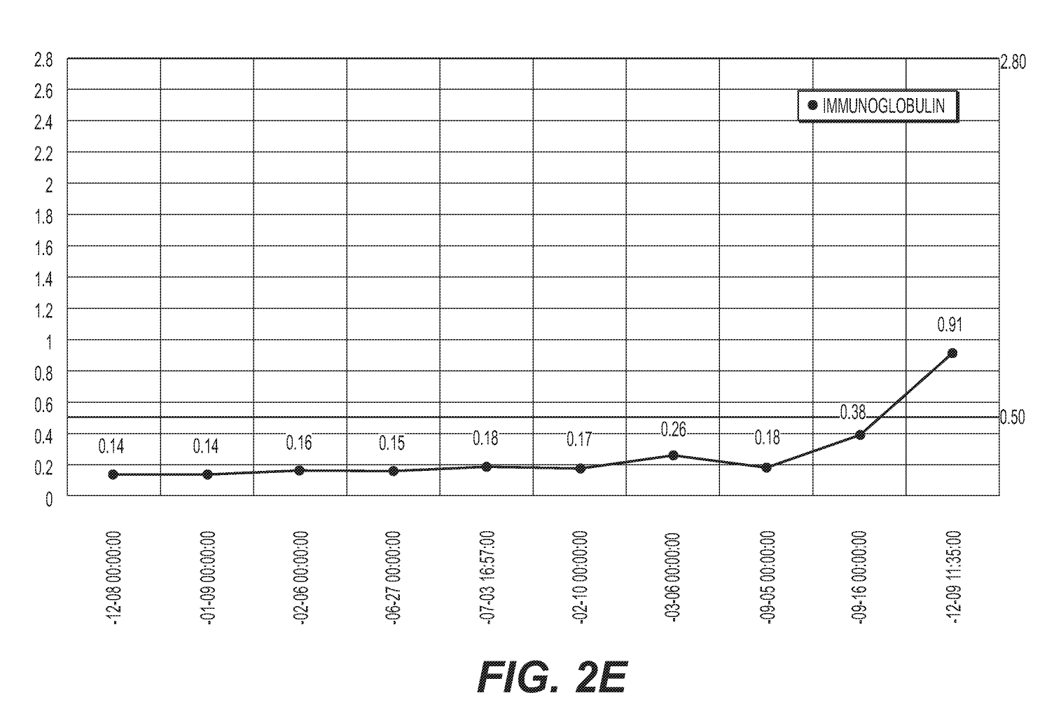

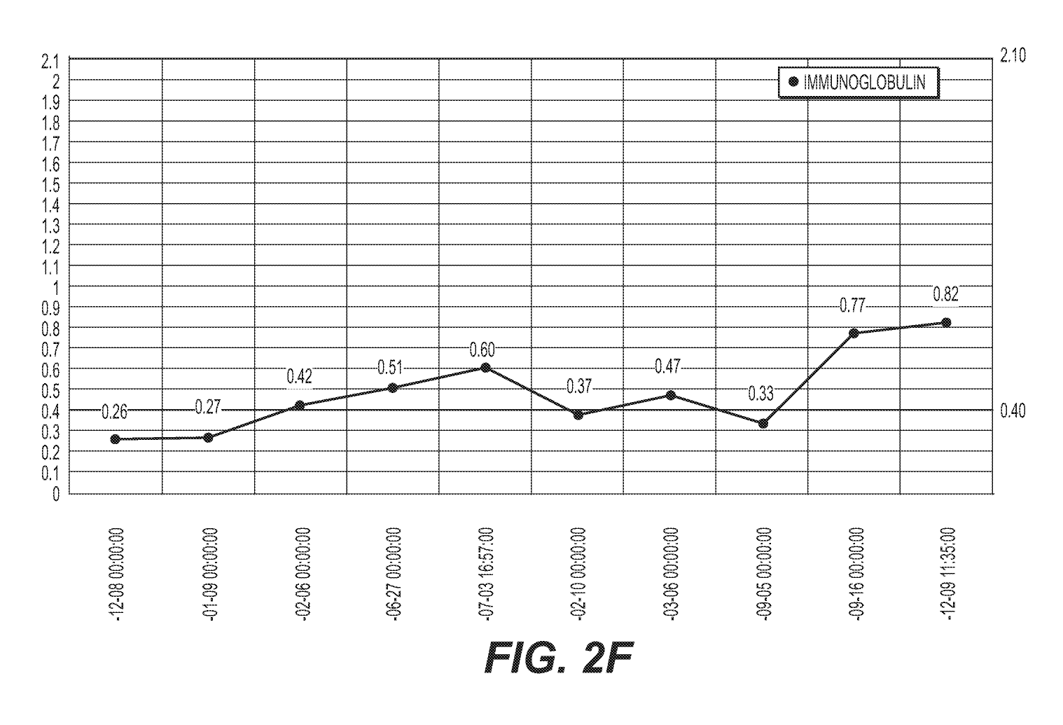

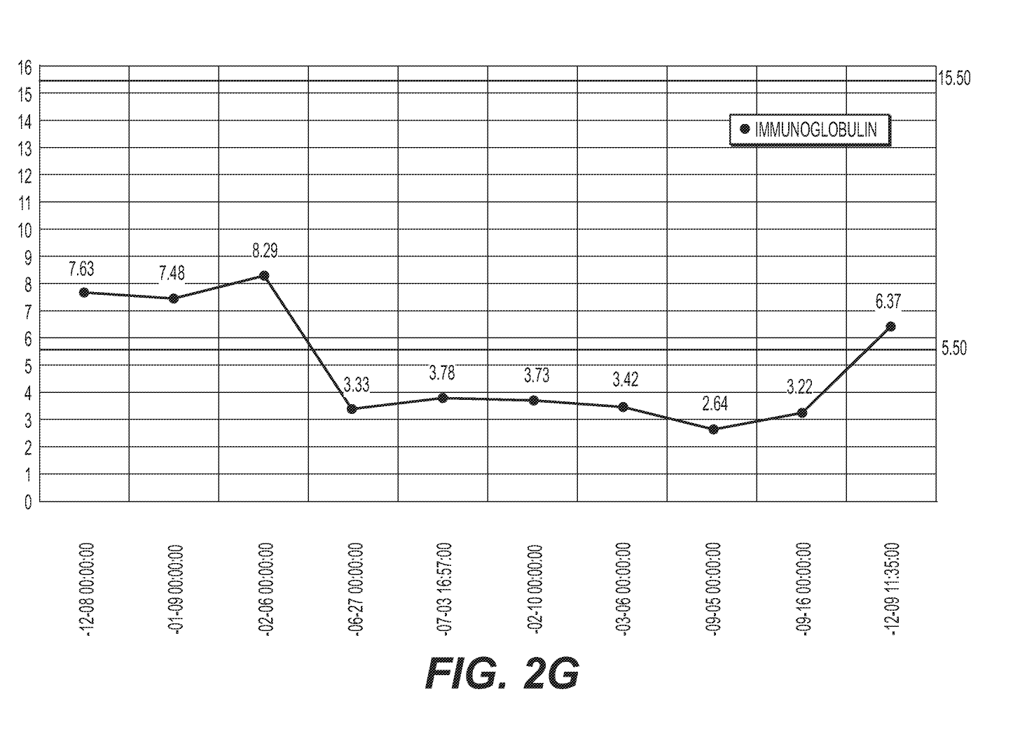

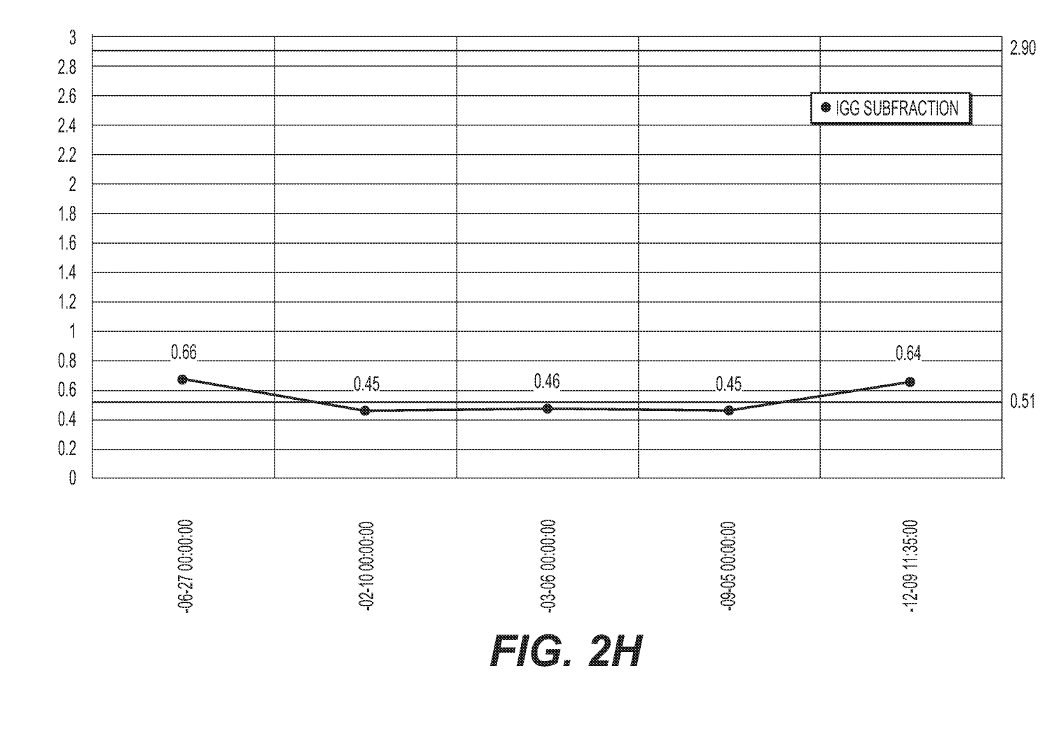

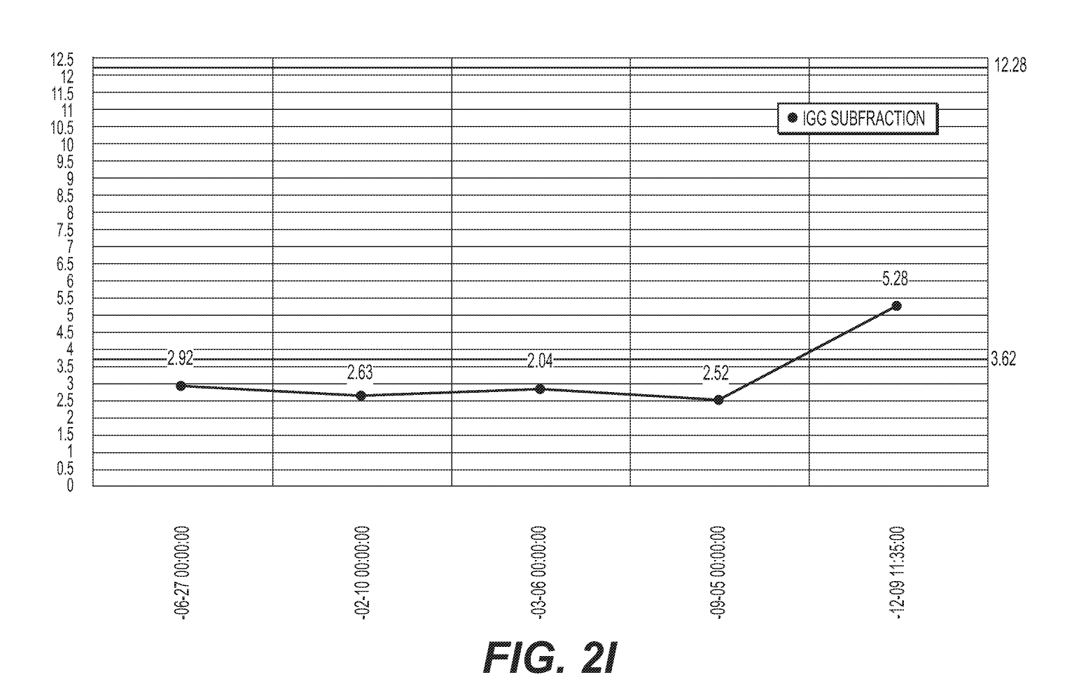

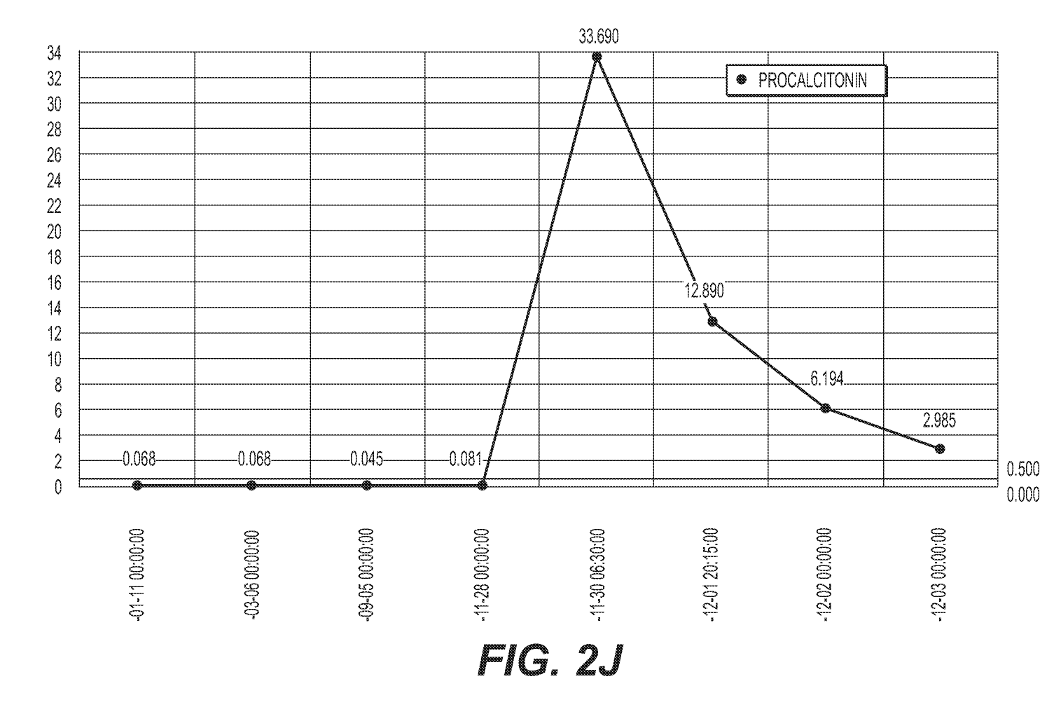

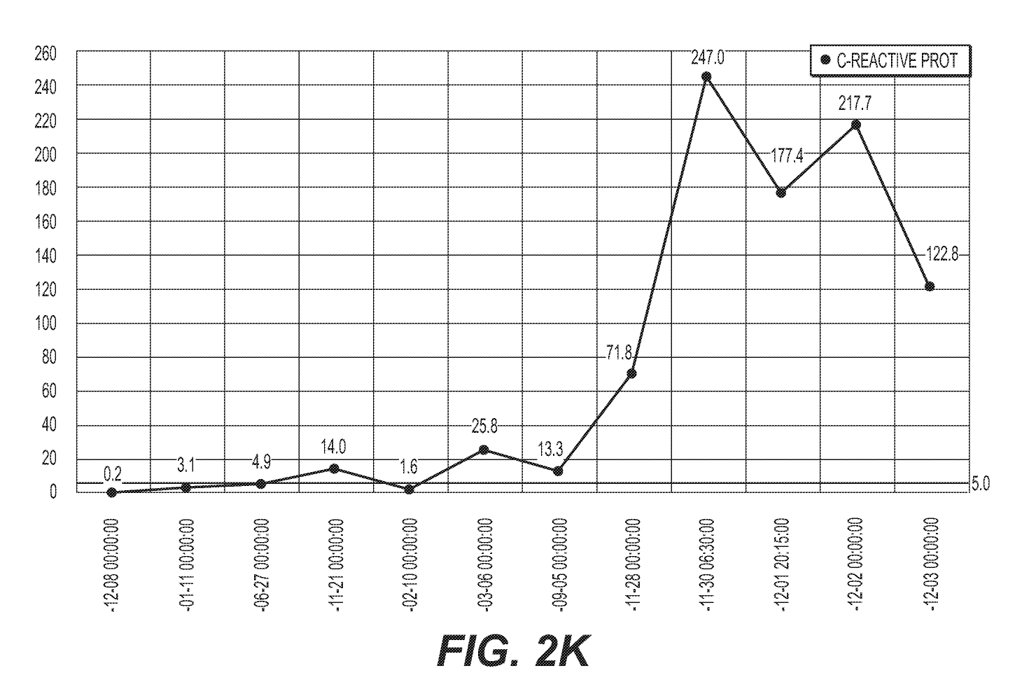

Patient 201 was administered NPIS40 according to Example 5. The blood levels of various components were measured as shown in FIGS. 2A-2K. Briefly, patient 201 was on the NPiS40 since September, but around the 20 September patient 201 it is suspected that the patient may have “extended” the dosage frequency, in other words a dosage was skipped.

CD4 and CD8 curves (see FIGS. 2C and 2D) show that on 5 September both CD4 and CD8 were low. NPIS40 treatment was commenced on 5 September and subsequently both parameters rose rapidly, only to drop again after 20 September. NPIS40 was reintroduced on a more regular program and a rapid response was observed.

Patient 201 was admitted to the hospital around 25 November with a viral infection but subsequently developed septicemia. Upon testing, PCT rose rapidly to 33.9, a clear indication of septicemia. Neutrophil and White Blood Cell counts (WBC) rose and then settled down on IVI antibiotics (see FIGS. 2A and 2B). Bothe levels rose whereby CRP and acute phase protein remained aggressively high. Antibiotics were changed to more potent IVI antibiotics with a resultant decrease in both parameters as of 1 December.

A surprising result was the patient's response in terms of immunoglobulins (IG). IG parameters were low, except after treatment with intravenous IG until end of February (see FIG. 2E whereby IG curve had a value of 7.63 on 8 December. Thereafter patient 201 developed a rare neurological side-effect and the IVI IG was stopped. Total IgG dropped to 3.33 and only rose after the NPIS40 had been optimized (9 December) (see FIG. 2G). Both IgG sub-fractions (IgG1:5.28 and IgG2:0.64 rose above the minimum in a similar fashion (see FIG. 2H). The IgM (0.82) and IgA (0.91) followed suite. This result indicates that CD4 helper cells support the production of IgG subclasses, IgA and IgM.

### Example 8

### Comparison of CD4 and CD8 Levels

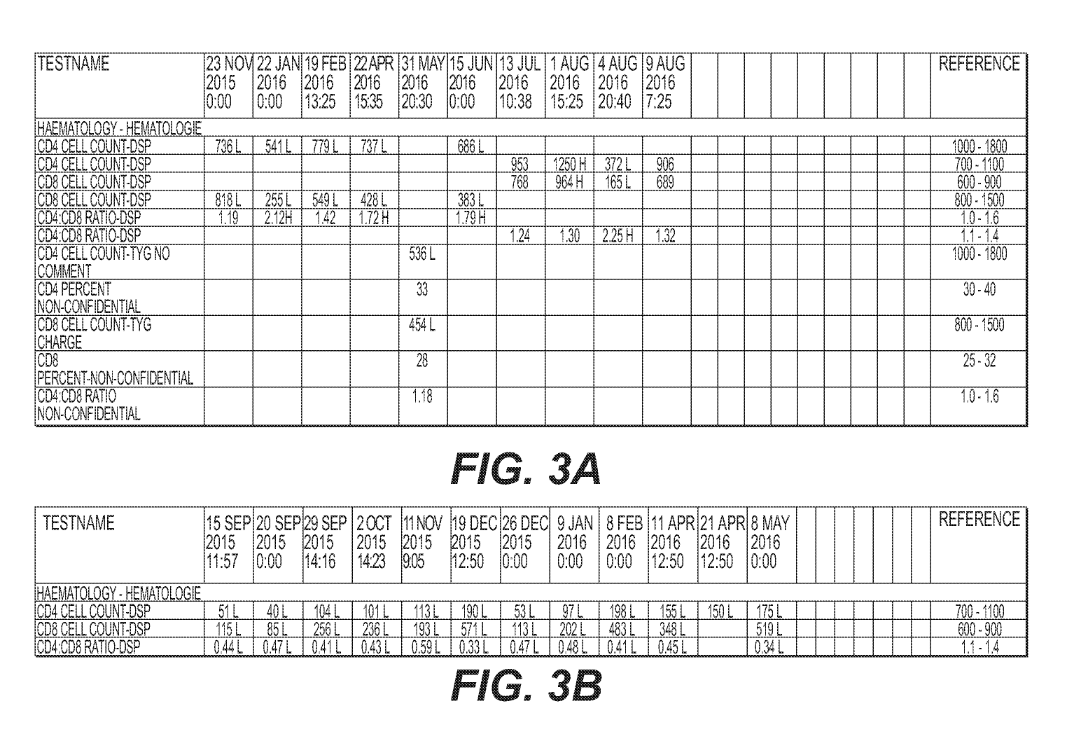

Shown in FIGS. 3A and 3B are blood results concerning the CD4 and CD8 counts and the response to NPiS40 for two patients.

As shown in FIG. 3A, the patient exhibited a sharp response between the 15 June, where after the NPiS40 was commenced and the 13 July. Likewise, on 2 August, the NPiS40 was not given. The patient subsequently developed septicemia and only after the antibiotic management and restarting NPiS40 by the 4 August, did the CD4 and CD8 cells respond positively.

As shown in FIG. 3B, NPiS40 injections were commenced on the 12 December and the Chemotherapy restarted on the 20 December. An initial positive response can be seen by 19 December and a subsequent drop thereafter because of the restart of the chemotherapy and finally followed by a slow increase till May.

### Example 9

### Analysis of Cytokine Levels

Different formulations of the serum-derived NPIS40, collected as set forth in Example 1, were tested as therapeutic agents. Product was derived from serum via tangential flow filtration process. The end product, referred to as NPIS40, was analyzed via mass spectral analysis which shows the main component to be primary peptides as set forth in Example 4.

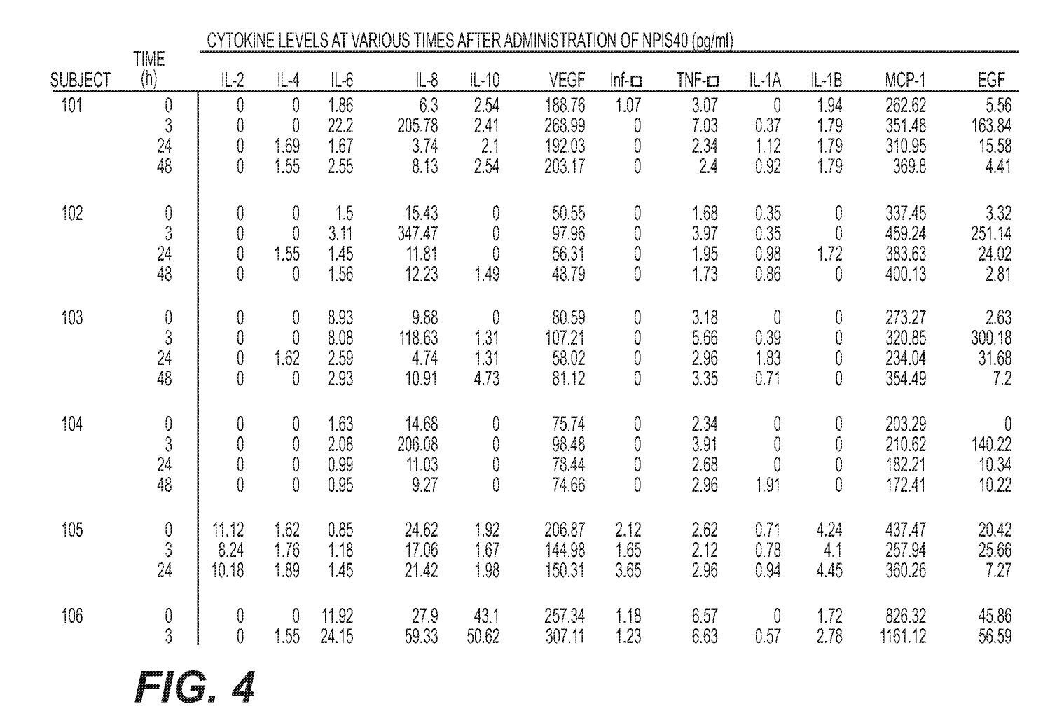

Volunteers were administered doses of NPIS40 equivalent to a 1×, 2× and 3× provision of test agent. A test bank of cytokines was monitored over time: Baseline (0 hour) and 3, 24, and 48 hours after administration. The data obtained with regard to the cytokines analyzed is shown in FIG. 4 and Table 2, which lists the levels of 11-8 after administration of NPIS40.

TABLE 2
IL-8 levels at Various Times After Administration of NPIS40

| IL-8 | 0 hr | 3 hr | 24 hr | 48 hr |
|---|---|---|---|---|
| 1x | 6.3 | 205.78 | 3.74 | 8.13 |
| 2x | 15.43 | 347.47 | 11.81 | 12.23 |
| 3x | 9.88 | 118.63 | 4.74 | 10.91 |

Value readings of IL-1-betta, Interferon-gamma and IL-2 were at or near zero for all times measured after administration as there was no apparent profile modulation. However, IL-4, IL-6, IL-8, VEGF, TNF-α, MCP-1, and EGF all showed an increase in serum concentration at 3 hours followed by a return to baseline. TNF-alpha showed a near doubling in concentration and release tended to be a signal initiator for downstream effects. Its expression is typically very short. IL-10 data was not consistent, but in general showed a delay in peak serum concentrations with respect to the 3-hour time point. IL-10 is known to assist in suppressing inflammatory cytokine expression (e.g., IL-6). CD4 and CD8 are upregulated/sustained in cancer patients being treated with high doses of chemotherapeutic drugs.

### Example 10

### Chemistry Profile of a Treated Patient

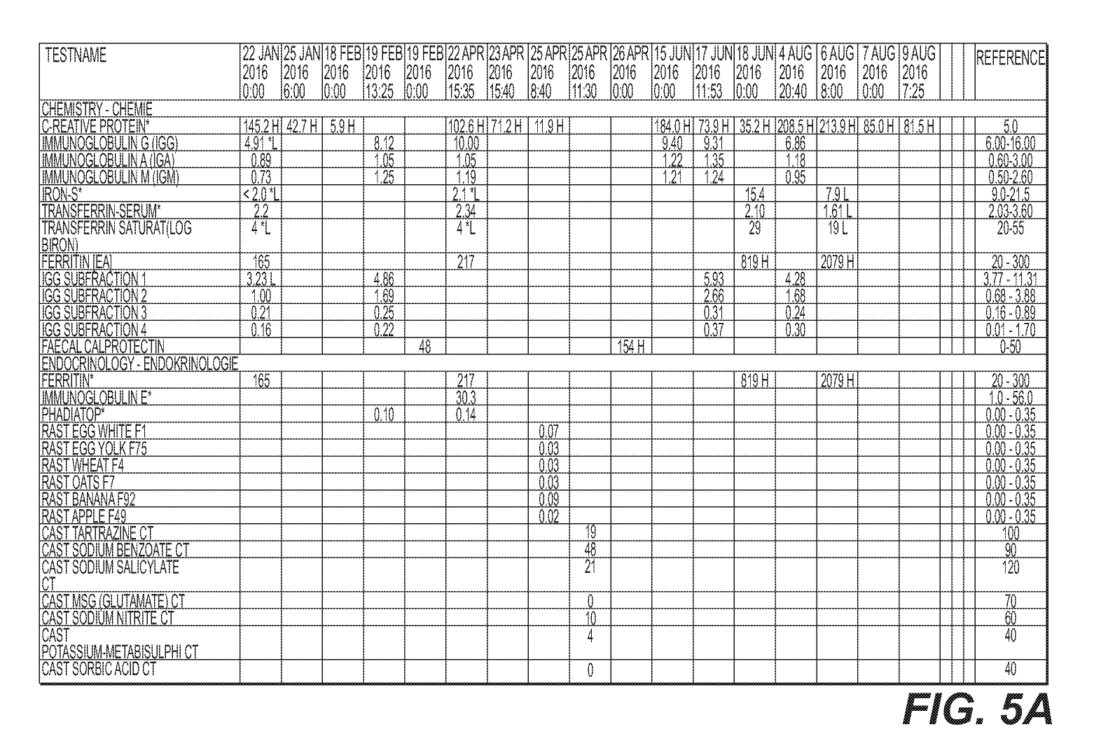

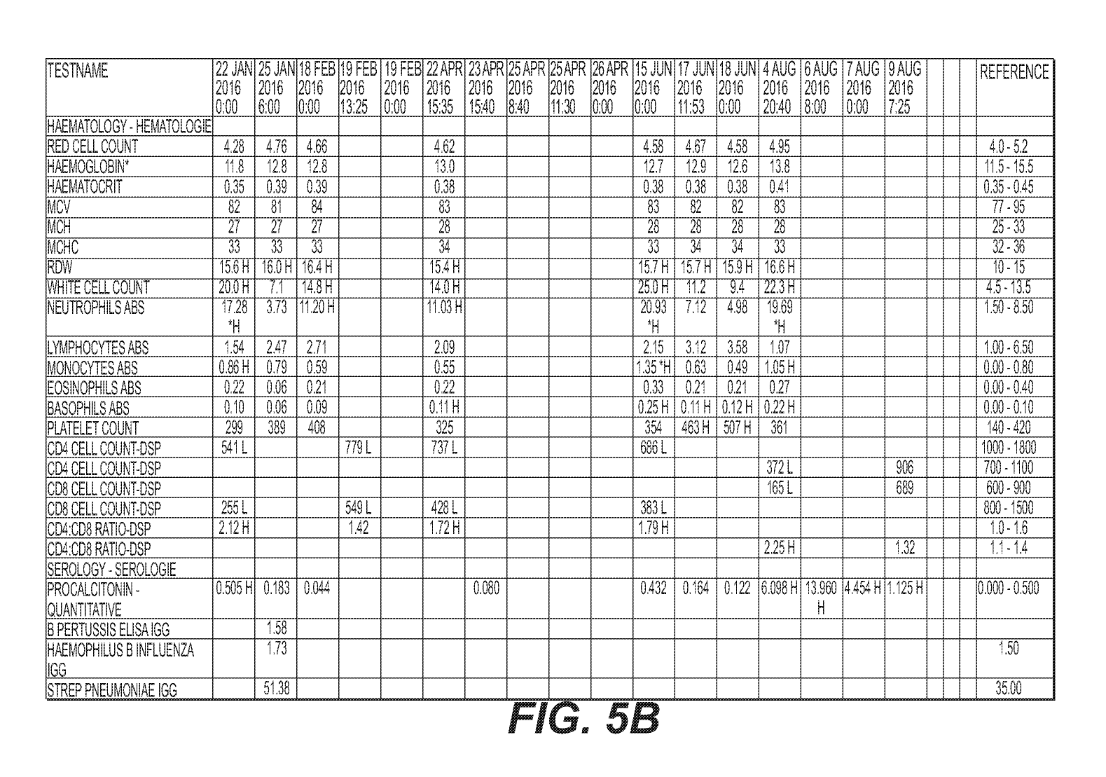

As shown in FIGS. 5A and 5B, the chemistry profile of a single patient treated with NPIS40. The iron stores normalized after the beginning of treatment with NPIS40. Due to poor compliance, iron stores dropped 2 August. The role of hepcidin released by gut macrophages is demonstrated.

### Example 11

### Clinical Observations of Treated Patients

Within 3 hours of an injection of NPIS40 there is a multi-fold up-regulation of IL-6, which is the bio-marker for the initiation of an Innate Immune response. After approximately 24 hours there is a multi-fold up-regulation of 1L-10, which is both, and adaptive immune bio-marker as well as the “switch,” that downregulates stemming the inflammatory cascade. The cascade of multiple cytokines as INF-gamma, TNF-α, GM-CSF, and many others; most importantly in regard to Autism, MCP-1. These cascades indicate that a) NPIS40 modulates both the adaptive and innate immune systems. No other molecule or medication modulates both systems. b) The other cytokine cascades are indicative of modulating proper cell signaling, and cellular communication that gets disrupted in chronic inflammation. c) The relieving of chronic inflammation which is the harbinger of most diseased states cannot be underestimated. d) The up-regulation of MCP-1, which has now explained the observed and improved neurological conditions of Multiple Sclerosis (MS), ALS, Parkinson's, Alzheimer, PLS, Autism, and others. These improved conditions were anecdotal, as they were done under informed consent or a compassion waiver application for other conditions where these symptoms were relieved as well. Immediate and lasting improvements were seen in seven out of seven autistic children. By identifying the up-regulation of MCP-1 after the administration of NPIS40, the positive effects of Monocyte Chemoattractant Protein 1 can be understood. The short definition of MCP-1 is: The chemokine (C-C motif) ligand 2(CCL2), is also referred to as monocyte chemoattractant protein 1 (MCP1), and small inducible cytokine A2. CCL2 is a small cytokine that belongs to the CC chemokine family. CCL2 acts to recruit monocytes, memory T cells, and dendritic cells to the sites of inflammation produced by either tissue injury or infection. The most important feature of this recruitment of cells is that the monocytes, memory T cells, and dendritic cells can and do pass through the blood/brain barrier (BBB). This is a major breakthrough as the standard of care in many neurological inflammatory conditions are the application of steroids, which have been proven to be unable to pass through the blood brain barrier.

The immune system is thought to play an important role in autism; children with autism have been found by researchers to have inflammation of both the peripheral and central immune systems as indicated by increased levels of pro-inflammatory cytokines, and significant activation of microglia. Biomarkers of abnormal immune function have also been associated with increased impairments in behaviors that are characteristic of the core features of autism, such as deficits in social interactions and communication. Interactions between the immune system and the nervous system begin early during the embryonic stage of life, and successful neurodevelopment depends on a balanced immune response. It is thought that activation of a pregnant mother's immune system; such as from environmental toxicants or infection can contribute to causing autism through causing a disruption of brain development. This is supported by recent studies that have found that infection during pregnancy is associated with an increased risk of autism.

The secondary factor, but as important in these immune cells being recruited to the site of inflammation and injury in the brain, is their function in the important operation of the Glymphatic system. The glymphatic system (or glymphatic clearance pathway) is a functional waste clearance pathway for the vertebrate central nervous system (CNS). The pathway consists of a para-arterial influx route for cerebrospinal fluid (CSF) to enter the brain parenchyma, coupled to a clearance mechanism for the removal of interstitial fluid (BF), and extracellular solutes from the interstitial compartments of the brain and spinal cord.

The name “glymphatic system” was coined by the Danish neuroscientist Maiken Nedergaard in recognition of its dependence upon glial cells, and the similarity of its functions to those of the peripheral lymphatic system. Two articles by Louveau et al. from the University of Virginia School of Medicine and Aspelund et al. from the University of Helsinki, reported independently the discovery that the dural sinuses and meningeal arteries are in fact lined with conventional lymphatic vessels, and that this long-elusive vasculature forms the connecting pathway for the entrance and exit of lymphatic fluid, and immune cells from the meningeal compartment to the glymphatic system.

NPIS40 triggers a cytokine cascade involving MCP-1 which potentiates a restoration of a healthy homestatic state—resolving chronic cellular inflammation, enabling the recruitment of healing immune cells through the BBB and the clearance of damaged tissue from the brain. Collectively, these actions provide therapeutic benefits to an Autistic child.

### Example 12

### Amino Acid Sequences of BRMs of the Invention

Amino acid sequences that function as BRMs of the invention include one or more of:

(fragment of Fibrinogen B)
SEQ ID NO. 1
YLDYDEVDDNRAKLPLDA
(fragment of Fibrinogen B)
SEQ ID NO. 2
LDYDEVDDNRAKLPLDA
(fragment of Fibrinogen B)
SEQ ID NO. 3
GYLDYDEVDDNRAKLPLD
(fragment of Fibrinogen B)
SEQ ID NO. 4
GYLDYDEVDDNRAKLPLDA
(fragment of Fibrinogen B)
SEQ ID NO. 5
GYLDYDEVDDNRAKLPLDAR
(fragment of Fibrinogen A)
SEQ ID NO. 6
FLAEGGGV
(fragment of Fibrinogen C terminal)
SEQ ID NO. 7
SEETKENERFTV
(fragment of Fibrinogen C terminal)
SEQ ID NO. 8
SAKFSAEEELEIR
(fragment of Fibrinogen A)
SEQ ID NO. 9
DEAESIEDLGIKGAHATKTGHA
(fragment of Fibrinogen A)
SEQ ID NO. 10
DEAESIEDLGIKGAHATKTGHAKA
(fragment of Fibrinogen A)
SEQ ID NO. 11
IQTADDSDPVGGEFLAEGGGV
(fragment of Fibrinogen A)
SEQ ID NO. 12
LCLVLSLVGAIQTADDSDPVGGEFLAEGGGV

These sequences may be purified from mammalian sources such as serum or artificially synthesized. These sequences may be modified such as, for example, the first and/or second tyrosine (Y) may be phosphorylated (HPO₃), sulfonated (SO₃), and/or modified with a 3-nitrotyrosine, 3-aminotyrosine, 3,4-dihydroxyphenylalanine, 3,3′-dityrosine and other cross-links, 3-chlorotyrosine, 3,5-dichlorotyrosine (DiClY), and/or quinone imine. Sequences may be modified by bromination and/or iodination. Sequences may preferably contain substitutions at, for example, D to E, V to I or L, N to Q, and/or G to A.

### Example 13

### Miscellaneous Patient Experiences

NPIS40 was administered sub-subcutaneously to three patients weekly. Patient one responded with a three-fold increase in both CD4 and CD8. Patent two normalized both CD4 and CD8 within three weeks which remained stable for at least four months. Patient two had 52 prior hospital admissions for septicemia by age 6. Patient three had multiple prior recurrent infections and deficiencies in IgA, IgG, CD4 and CD8. CD4 and CD8 values remained low after administration of NPIS40. Patient three was hospitalized with septicemia during the trial, which allowed for daily testing of CD4 and CD8 values and further treatment. After further administration of NPIS40, CD4 and CD8 values stated to decrease at day 4.

BRM is administered via I.V. injection weekly to an adult male patient. Within 30 minutes to an hour of administration, TNF-α is up-regulated initiating the release of IL-6. This is followed by up regulation of macrophages and NK cells. At about 12 to 30 hours post administration, a multifold up regulation of IL-10 brings down the pro-inflammatory cytokine actions of IL-6 and pro-inflammatory TH1 cells. IFN-gamma which possesses anti-viral properties and other important immunoregulatory functions along with granular macrophage-colony stimulating factor (GM-CSF) (under evaluation for treating neurological diseases) as well as monocyte chemoattractant protein (MCP-1). MCP-1 stimulates the recruitment of T-cells B-cells, macrophages and other immune cells to the sites of injury and damage to initiate the removal of damaged tissue. Associated with the effect of the up-regulating MCP-1 is that those cells can pass through the BBB which assists in the healing of many neurological conditions. IL-8, vascular endothelial growth Factor (VEGF) and Epidermal Growth Factor (EGF) are upregulated subsequent to the initial pro-inflammatory spike. IL-8 and VEGF are potent promoters of angiogenesis, and EGF is a potent promoter of cell growth, proliferation and differentiation. Collectively these provide a powerful stimulus for wound healing effects.

The cascade is completed in about 120 hours. If additional provision of the invention is deemed necessary (e.g., to continue assisting in restoring normalized homeostasis) administrative practices can be continued.

### Example 14

### BRM Analysis

BRM was analyzed and modified forms of SEQ ID No 1 and SEQ ID NO 4 were found with modifications on the first or second tyrosine which may be sulfations or phosphorylations. The mass difference is less than 0.01 Da, and therefore nearly impossible to determine by mass spec analysis. Two fragments of C3 were present at about 0.1% of the fibrinogen beta components, SEQ ID NO 7 and SEQ ID NO 8, and two fragments from fibrinogen alpha at about 0.01%, SEQ ID NO 11 and SEQ ID NO 9. Samples are analyzed for glycans and oligonucleotides, undetectable by mass spec analysis.

A lectin-based enrichment was performed and followed by HILIC LC-MS and a second nasospray infusion was performed in negative mode. Neither assay generated a response above background and, accordingly, either no oligonucleotide was present or any present was below detectable levels.

Bioactivity was assayed via a murine salmonellosis model. Both the modified and unmodified peptides failed to produce a marked response in the salmonellosis challenge assay.

### Example 15

### Dosage Profiles

Pharmaceutical compositions that constitutes BRM do not always conform to standards of dosage regimens as for other pharmaceuticals such as cytotoxic drugs. Product of the invention is maintained at minus 20 C until use. Before use, product is allowed to thaw to room temperatures, or about 20° C. No autoclaving or external heat sources are involved. Doses are typically packaged in 1.5 ml aliquots and each vial is a complete dose. What changes, per chronic condition, treatment protocol, and duration, is the frequency of the administration of the dose. In most chronically diagnosed conditions, the frequency is: 1 dose every 3 days for at least the first month, possibly for 3 months, with frequency being extended to once every 5 days, then once per week, then twice per month, to a maintenance dose of once per month, or until symptoms subside. For patients suffering from chronic or debilitating conditions, subcutaneous injections of product are preferred. The patient's cytokine activity is monitored during treatment to determine a return to homeostasis or otherwise normal functioning of their Innate and/or adaptive immune systems. Occasionally, such as with prolonged debilitative chronic conditions, the patient may remain on treatment for much longer periods, even years.

New patients receiving their first injectable dose use the following protocol: 0.5 ml of the dose injected sub-cutaneously. If no allergic reaction or anaphylaxis is observed in about 20-30 minutes, the remainder of the dose is administered sub-cutaneously. Preferred injection sites are the abdomen, the arm, the upper hip, the exterior thigh. Dosages are generally as follows:

### Frequency by Condition

Length of treatment may vary depending on severity of condition.

Concentrations:

A. Chronic (C)—900 to 1100 μg per 1.5 ml

B. Intensive (I)—750-850 μg per 1.5 ml

C. Therapeutic (T)—450-600 μg per 1.5 ml

D. Maintenance (M)—300-380 μg per 1.5 ml

Frequency for Recalcitrant Diabetic Wounds and/or Pressure Wounds due to immobility

A. Every 3 days for the first month, Chronic (C)

B. Every 5 days for next six doses, Intensive (I)

C. One dose per week until the wound closes, Therapeutic(T)

D. One dose per week for 4 weeks. Maintenance(M)

### Frequency for Chronic Lyme Disease

A. Every 3 days for the first eight doses, C

B. Every 5 days for next twelve doses, I

C. One dose per week for the next 8 doses, T

D. One dose every 10 days for 6 doses. M

### Frequency for Primary Progressive Multiple Sclerosis

A. One dose every 3 days for 3 months C

B. Once per week for 6 months T

At 9 months, new panels of blood work and neurological testing based upon test findings additional protocol to be administered. This protocol is generally similar for inflammatory and demyelinating conditions as well CIDP (chronic inflammatory demyelinating polyneuropathy), Guillain-Barre Syndrome, Progressive Inflammatory Neuropathy, Diabetic Neuropathy and ALS (Amyotrophic Lateral Sclerosis) including CMT (Charcot-Marie-Tooth). Nutritional counseling focusing on an anti-inflammatory diet is strongly recommended.

### Frequency for Malignant Neoplasm

A. One dose every 3 days for 2 months C

B. Every 5 days for the next 8 injections T

C. Once per week for 3 months M

D. Appropriate Blood panels per condition every 90 days, with reassessment of protocol.

### Frequency for Reflex Sympathetic Dystrophy (RSD) and Hyper-Analgesia

A. One dose every 3 days for one month C

B. Every five days for the next 8 injections I

C. Once per week for 3 months T

D. As necessary to maintain pain relief. M

### Frequency for Chronic Inflammatory Conditions as in Rheumatoid Arthritis, Arteriosclerosis, Ischemia, Chronic Psoriasis, Irritable Bowel Syndrome (IBS), Including Inflammatory Diseases Like Crohn's Disease and Ulcerative Colitis

A. One dose every 3 days for two months

B. Every 5 days for two months

C. Once per week for 3 months

Frequency for Autoimmune Disease which Includes any Number of Over One Hundred Named Conditions.

Acute:

A. One dose every 3 days for 5 weeks C

B. Every 5 days for 8 weeks I

C. Once per week for 3 months T/M

Chronic:

A. One dose every 3 days for 9 weeks C

B. Every 5 days for 9 weeks I

C. Once per week for 9 weeks T

Corticosteroids and any other steroid therapy can negate the positive effects of product of the invention and all steroid therapies should be discontinued at least 30 days prior to the start of any protocol.

Other embodiments and uses of the invention will be apparent to those skilled in the art from consideration of the specification and practice of the invention disclosed herein. All references cited herein, including all publications, and all U.S. and foreign patents and patent applications are specifically and entirely incorporated by reference. The term comprising, where ever used, is intended to include the terms consisting and consisting essentially of. Furthermore, the terms comprising, including, and containing are not intended to be limiting. It is intended that the specification and examples be considered exemplary only with the true scope and spirit of the invention indicated by the following claims.

1.-11. (canceled)

12. A method of manufacturing a pharmaceutical composition comprising:
providing a mammalian serum that is sterile;
mixing equal parts of the sterile serum with a sterile 0.5% saline solution to form a mixture;
passing the mixture through tangential flow chromatography followed by molecular weight cut-off dialysis and filtration through a 0.2 μm filter forming a filtered solution;
apportioning the filtered solution into single dose vessels; and
freezing the filtered solution at minus 10° C. or less.

13. The method of claim 12, wherein the molecular weight cut-off dialysis is 10 kDa cut-off dialysis.

14. The method of claim 12, wherein the single dose vessels comprises from about 0.5-5 ml each.

15. The method of claim 14, wherein the single dose vessels comprises from about 0.1 μg/ml to about 100 μg/ml of a peptide of the sequence of SEQ ID NO 1 and/or SEQ ID NO 4.

16. The method of claim 12, wherein the filtered solution is tested for the presence of endotoxin.

17. The method of claim 12, which is performed under GMP standards for pharmaceutical compositions.

18.-35. (canceled)

36. The method of claim 12, wherein the mammalian serum comprises goat serum.

37. The method of claim 12, wherein the mammalian serum is free of Mycoplasma.

38. The method of claim 12, wherein the mammalian serum or the filtered solution is treated with a chemical or physiological process such that one or more functional groups therein are modified.

39. The method of claim 38, wherein the chemical or physiological process creates a free hydroxy, free amino and/or free mercapto group.

40. The method of claim 38, wherein the filtered solution contains SEQ ID NO 1 and/or SEQ ID NO 4 and the chemical or physiological process creates a modification that is a sulfation and/or a phosphorylation of the first and/or second tyrosine of SEQ ID NO 1 and/or SEQ ID NO 4.

41. The method of claim 12, wherein the filtered solution contains SEQ ID NO 1 and/or SEQ ID NO 4.

42. The method of claim 41, wherein the filtered solution contains no fibrin peptide sequences other than peptide that contain SEQ ID NO 1 and/or SEQ ID NO 4.

43. The method of claim 12, wherein the filtered solution contains one or more peptides that contain one or more of SEQ ID NO 5, 6, 7, 8, 9, and 11.

44. The method of claim 12, wherein the filtered solution is aqueous and formulated for intravenous administration.

45. The method of claim 12, wherein the filtered solution contains no detectable oligosaccharide.

46. The method of claim 12, wherein the filtered solution contains no detectable glycan.

47. The method of claim 12, wherein the single dose vessels contain about 0.5-5 ml which contain from about 0.1 μg/ml to about 100 μg/ml of peptide.

48. The method of claim 12, further comprising adding one or more pharmaceutically acceptable agents to the filtered solution.

49. The method of claim 47, wherein the one or more pharmaceutically acceptable agents are selected from the group consisting of water, oil, edible oil, fatty acids, lipids, polysaccharides, cellulose, glycerin, glycol, and combinations thereof.

50. The method of claim 16, wherein the presence of endotoxin per single dose vessel is insufficient to promote a pyrogenic response when administered to a patient.


SEQUENCE LISTING (12 sequences)

SEQ ID NO: 1 - protein, 18 aa, Artificial Sequence
  Description of Artificial Sequence Synthetic peptide
YLDYDEVDDNRAKLPLDA

SEQ ID NO: 2 - protein, 17 aa, Artificial Sequence
  Description of Artificial Sequence Synthetic peptide
LDYDEVDDNRAKLPLDA

SEQ ID NO: 3 - protein, 18 aa, Artificial Sequence
  Description of Artificial Sequence Synthetic peptide
GYLDYDEVDDNRAKLPLD

SEQ ID NO: 4 - protein, 19 aa, Artificial Sequence
  Description of Artificial Sequence Synthetic peptide
GYLDYDEVDDNRAKLPLDA

SEQ ID NO: 5 - protein, 20 aa, Artificial Sequence
  Description of Artificial Sequence Synthetic peptide
GYLDYDEVDDNRAKLPLDAR

SEQ ID NO: 6 - protein, 8 aa, Artificial Sequence
  Description of Artificial Sequence Synthetic peptide
FLAEGGGV

SEQ ID NO: 7 - protein, 12 aa, Artificial Sequence
  Description of Artificial Sequence Synthetic peptide
SEETKENERFTV

SEQ ID NO: 8 - protein, 13 aa, Artificial Sequence
  Description of Artificial Sequence Synthetic peptide
SAKFSAEEELEIR

SEQ ID NO: 9 - protein, 22 aa, Artificial Sequence
  Description of Artificial Sequence Synthetic peptide
DEAESIEDLGIKGAHATKTGHA

SEQ ID NO: 10 - protein, 24 aa, Artificial Sequence
  Description of Artificial Sequence Synthetic peptide
DEAESIEDLGIKGAHATKTGHAKA

SEQ ID NO: 11 - protein, 21 aa, Artificial Sequence
  Description of Artificial Sequence Synthetic peptide
IQTADDSDPVGGEFLAEGGGV

SEQ ID NO: 12 - protein, 31 aa, Artificial Sequence
  Description of Artificial Sequence Synthetic polypeptide
LCLVLSLVGAIQTADDSDPVGGEFLAEGGGV